SEQUENCE LISTING (36 sequences)

SEQ ID NO: 1 - protein, 499 aa, Human immunodeficiency virus type 1
  misc_feature (82)..(82): Xaa can be any naturally occurring amino acid
MGARASVLSGGKLDAWEKIRLRPGGKKKYRIKHLVWASRELERFALNPGLLETTEGCQQILEQLQPTLRTGTEEIKSLYNAXATLYCVHQRIEVKDTKEALEEVEKIQKKSQKKTQQAAMGEGNSSQVSQNYPIVQNAQGQMVHQPLSPRTLNAWVKVVEEKAFNPEVIPMFSALSEGATPQDLNTMLNTVGGHQAAMQMLKDTINEEAAEWDRTHPPQAGPIPPGQIREPRGSDIAGTTSNLQEQIRWMTSNPPIPVGEIYKRWIILGLNKIVRMYSPVSILDIRQGPKEPFRDYVDRFFKTLRAEQATQEVKNWMTDTLLVQHANPDCKTILRALGPGATLEEMMTACQGVGGPGHKARVLAEAMSQASNSAAAIMMQKGNFKGPRRIKCFNCGKEGHLARNCRAPRKKGCWKCGKEGHQMKDCTERQANFLGKIWPSNKGRPGNFLQNRPEPTAPPAESFGFGEEIAPSPKQAPKQEPKGELYPLASLKSLFGNDP

SEQ ID NO: 2 - protein, 497 aa, Human immunodeficiency virus type 1
MGARASVLSGGKLEAWEKIRLRPGGKKKYRMKHLVWASRELERFALNPGLLETAEGCQQIIEQLQPTLKTGSEELKSLFNTVATLWCVHQKVDVKDTKEALDKIEEVQNESQQKTQQAAAGTGSSSKVSQNYPIVQNAQGQMVHQPLSPRTLNAWVKVVEEKGFKPEVIPMFSALSEGATPQDLNMMLNIVGGHQAAMQMLKETINEEAAEWDRVHPVHAGPIPPGQMREPRGSDIAGTTSTLQEQIGWMTGNPAIPVGDIYKRWIILGLNKIVRMYSPASILDIRQGPKEPFRDYVDRFYKTLRAEQATQEVKNWMTETLLVQNANPDCKSILRALGPGATLEEMMTACQGVGGPSHKARVLAEAMSQAQHTNILMQRGNFKGQKRIKCFNCGKEGHLARNCKAPRKKGCWKCGKEGHQMKDCTERQANFLGKIWPSNKGRPGNFPQNRLEPTAPPAENWERGEEMTPLPKQEQKNKDPPPLVSLKSLFGNDPLSQ

SEQ ID NO: 3 - protein, 6 aa, Artificial
  6x HIS Peptide tag
HHHHHH

SEQ ID NO: 4 - protein, 10 aa, Artificial
  c-MYC Peptide tag
EQKLISEEDL

SEQ ID NO: 5 - protein, 9 aa, Artificial
  HA Peptide tag
YPYDVPDYA

SEQ ID NO: 6 - protein, 11 aa, Artificial
  VSV-G Peptide tag
YTDIEMNRLGK

SEQ ID NO: 7 - protein, 11 aa, Artificial
  HSV Peptide tag
QPELAPEDPED

SEQ ID NO: 8 - protein, 14 aa, Artificial
  V5 Peptide tag
GKPIPNPLLGLDST

SEQ ID NO: 9 - protein, 8 aa, Artificial
  FLAG Peptide tag
DYKDDDDK

SEQ ID NO: 10 - protein, 7 aa, Artificial
  SV40 latge T antigen nuclear localisation signal
PKKKRKV

SEQ ID NO: 11 - protein, 16 aa, Artificial
  Nucleoplasmin nuclear localisation signal
KRPAATKKAGQAKKKK

SEQ ID NO: 12 - protein, 7 aa, Artificial
  p54 nuclear localisation signal
RIRKKLR

SEQ ID NO: 13 - protein, 5 aa, Artificial
  SOX9 nuclear localisation signal
PRRRK

SEQ ID NO: 14 - protein, 9 aa, Artificial
  NS5A nuclear localisation signal
PPRKKRTVV

SEQ ID NO: 15 - protein, 96 aa, Human immunodeficiency virus type 1
MEQAPEDQGPQREPHNEWTLELLEELKNEAVRHFPRIWLHGLGQHIYETYGDTWAGVEAIIRILQQLLFIHFRIGCRHSRIGVTRQRRARNGASRS

SEQ ID NO: 16 - protein, 78 aa, Human immunodeficiency virus type 1
QREPHNEWTLELLEELKNEAVRHFPRIWLHGLGQHIYETYGDTWAGVEAIIRILQQLLFIHFRIGCRHSRIGVTRQRR

SEQ ID NO: 17 - protein, 87 aa, Human immunodeficiency virus type 2
MTEAPTEFPPEDGTPRRDLGSDWVIETLREIKEEALRHFDPRLLIALGYYIHNRHGDTLEGARELIKTLQRALFVHFRAGCNRSRIG

SEQ ID NO: 18 - protein, 112 aa, Simian immunodeficiency virus
MSDPRERIPPGNSGEETIGEAFEWLNRTVEEINREAVNHLPRELIFQVWQRSWEYWHDEQGMSPSYVKYRYLCLIQKALFMHCKKGCRCLGEGHGAGGWRPGPPPPPPPGLA

SEQ ID NO: 19 - DNA, 24 bp, Artificial
  C1221 DNA target
tcaaaacgtcgtacgacgttttga

SEQ ID NO: 20 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 21 - DNA, 320 bp, Human immunodeficiency virus type 1
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacat

SEQ ID NO: 22 - DNA, 324 bp, Human immunodeficiency virus
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaag

SEQ ID NO: 23 - DNA, 5900 bp, Artificial
  Sequence for Plasmid pBA Rev
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttggtaccgagctcggatccactagtaacggccgccagtgtgctggaattctgcagatatccatcacactggcggccgctcgagcatgcatctagactgccatggcaggaagaagcggagacagcgacgaagacctcctcaaggcagtcagactcatcaagtttctctatcaaagcaacccacctcccaaccccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaataggatccggctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattttaacaaaatattaacgtttacaatttaaatatttgcttatacaatcttcctgtttttggggcttttctgattatcaaccggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 24 - DNA, 320 bp, Vesicular stomatitis virus; identical to SEQ ID NO: 21

SEQ ID NO: 25 - DNA, 308 bp, Artificial
  Repair Matrix
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcat

SEQ ID NO: 26 - protein, 351 aa, Artificial
  I-SceI-HA-7/1-Vpr fusion protein
MHMKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLKYPYDVPDYAERQANFLGKIEQAPEDQGPQREPHNEWTLELLEELKNEAVRHFPRIWLHGLGQHIYETYGDTWAGVEAIIRILQQLLFIHFRIGCRHSRIGVTQQRRARNGASRS

SEQ ID NO: 27 - protein, 351 aa, Artificial
  I-SceI-HA-24/2-Vpr fusion protein
MHMKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLKYPYDVPDYAKARVLAEAMSEQAPEDQGPQREPHNEWTLELLEELKNEAVRHFPRIWLHGLGQHIYETYGDTWAGVEAIIRILQQLLFIHFRIGCRHSRIGVTQQRRARNGASRS

SEQ ID NO: 28 - protein, 342 aa, Artificial
  I-SceI-HA-G-Vpr fusion protein
MHMKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLKYPYDVPDYAIEQAPEDQGPQREPHNEWTLELLEELKNEAVRHFPRIWLHGLGQHIYETYGDTWAGVEAIIRILQQLLFIHFRIGCRHSRIGVTQQRRARNGASRS

SEQ ID NO: 29 - protein, 331 aa, Artificial
  I-SceI-HA-7/1 (delta 14-18) -Vpr fusion protein
MHMKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLKYPYDVPDYAERQANFLGKIPHNEWTLELLEELKNEAVRHFPRIWLHGLGQHIYETYGDTWAGVEAIIRILQQLLFIHFRIGCRHSRIGVTQQRR

SEQ ID NO: 30 - DNA, 22 bp, Artificial
  PCR primer
ccgccaccatgaccgagtacaa

SEQ ID NO: 31 - DNA, 20 bp, Artificial
  PCR primer
acgaagttatggtcaccgag

SEQ ID NO: 32 - DNA, 21 bp, Artificial
  PCR primer
ctcgtagaaggggaggttgcg

SEQ ID NO: 33 - protein, 354 aa, Artificial
  I-CreI single chain meganuclease
MANTKYNKEFLLYLAGFVDGDGSIIAQINPNQSSKFKHRLRLTFYVTQKTQRRWFLDKLVDEIGVGYVRDSGSVSQYVLSEIKPLHNFLTQLQPFLKLKQKQANLVLKIIEQLPSAKESPDKFLEVCTWVDQIAALNDSKTRKTTSETVRAVLDSLSGKKKSSPAAGGSDKYNQALSKYNQALSKYNQALSGGGGSNKEFLLYLAGFVDSDGSIIAQIKPRQSNKFKHQLSLTFAVTQKTQRRWFLDKLVDEIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 34 - protein, 9 aa, Artificial
  Description of Artificial sequence synthetic LAGLIDADG motif
LAGLIDADG

SEQ ID NO: 35 - protein, 10 aa, Artificial
  Description of artificial sequence protease cleavage site
ERQANFLGKI

SEQ ID NO: 36 - protein, 10 aa, Artificial
  Description of artificial sequence synthetic protease cleavage site
KARVLAEAMS

(19) United States
(12) Patent Application Publication
(10) Pub. No.: US 2012/0272348 A1
(43) Pub. Date: Oct. 25, 2012

(54) VIRAL VECTORS ENCODING A DNA REPAIR MATRIX AND CONTAINING A VIRION-ASSOCIATED SITE SPECIFIC MEGANUCLEASE FOR GENE TARGETING

(75) Inventors: Olivier Danos, New York, NY (US); Araksya Izmiryan, Paris (FR); Alix Bourdel, Vaureal (FR)

(73) Assignee: CELLECTIS, Paris (FR)

(21) Appl. No.: 13/384,468

(22) PCT Filed: Jul. 16, 2010
(86) PCT No.: PCT/IB2010/053246

§ 371 Date: Jul. 6, 2012

(30) Foreign Application Priority Data
Jul. 17, 2009 (IB) PCT/IB2009/006689

(51) Int. Cl. C12N 9/96; A01K 67/00; C12N 5/10; C12N 15/62; C12N 15/867; C12N 15/85

(52) U.S. Cl. 800/13; 435/188; 435/320.1; 435/455; 435/369; 435/358; 536/23.2

(57) ABSTRACT

The present invention relates to a fusion protein which comprises at least a functional meganuclease and a viral protein and in particular to fusion protein comprising at least a meganuclease, which recognises and cleaves a specific DNA target sequence and a viral peptide selected from the group Vpr and Vpx or a fragment or derivative thereof; wherein said fusion protein is able to associate with Lentivirus vector particles and following transduction into a host cell recognise and cleave said specific DNA target in vivo. The present Patent Application also relates to a viral particle comprising such a fusion protein and to the use of such fusion proteins and viral particles for gene targeting.

The present Patent Application relates to a fusion protein which comprises at least a functional meganuclease and a viral protein and in particular the fusion protein comprises a functional meganuclease and the Vpr or Vpx proteins or fragments thereof. The present Patent Application also relates to a viral particle comprising such a fusion protein and to the use of such fusion proteins and viral particles for gene targeting.

For many years a number of important pathological conditions have been known to be wholly or partially due to genetic causes, most normally mutations in the coding sequence of a gene which results in a modified gene product that is unable to perform its in vivo function and hence perturbs natural processes leading to the observed condition. In addition to mutations in the coding sequences, mutations can also occur in the regulatory regions of a gene.

The classical approach to dealing with such conditions is to treat the patient so as to alleviate as far as possible the observed condition, for instance Phenylketonuria which is an autosomal recessive genetic disorder is characterized by a deficiency of functional forms of the enzyme phenylalanine hydroxylase. Phenylalanine hydroxylase is necessary for the metabolism of the amino acid phenylalanine to the amino acid tyrosine. When the enzyme is deficient, phenylalanine accumulates and is converted into phenylpyruvate, the accumulation of phenylpyruvate causes problems with brain development and leads to progressive mental retardation, brain damage and seizures. Phenylketonuria is one of a few genetic diseases that can be controlled by diet. A diet low in phenylalanine and high in tyrosine is a very effective treatment, but there is currently no cure.

For the majority of genetic diseases the alleviation of symptoms is not possible or only partially successful using medicines or other treatments as without addressing the underlying genetic defect no meaningful alleviation of the pathology can occur.

In recent years many people have worked to develop and validate various techniques to cure genetic diseases in situ, these techniques being collectively known as gene therapy. In general, gene therapy involves the alteration of the genetic content of a cell so as to repair, remove or supplement the genetic defect which causes the disease.

Amongst a great many other technologies which have been developed to implement gene therapy treatments, artificial endonucleases with tailored specificities have been and continue to be developed for therapeutic purposes such as gene repair in monogenic diseases as well as for use in antiviral therapies and regenerative medicine. These tailored endonucleases can be used in direct gene targeting by inducing a homologous recombination (HR) event at the site of the faulty gene. This direct gene targeting approach has long been a major goal of gene therapy research and tailored site-specific endonuclease technologies are increasingly appearing to be the most feasible technological platform in which the goals of gene therapy can be achieved in a therapeutic setting [1].

Of the various types of artificial endonucleases proposed for use in gene therapy and other methods, meganucleases have emerged as a seemingly inexhaustible technological platform from which endonucleases to targets in any given sequence can be generated. In the wild, meganucleases are essentially represented by homing endonucleases. Homing Endonucleases (HEs) are a widespread family of natural meganucleases including hundreds of proteins [2]. These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus. Given their cleavage properties in terms of efficacy and specificity, they represent ideal scaffolds to derive novel, highly specific endonucleases.

HEs belong to four major families. The LAGLIDADG family, named after a conserved peptide motif found in the catalytic center, is the most widespread and the best characterized group. Whereas most proteins from this family are monomeric and display two LAGLIDADG motifs, a few have only one motif, and thus dimerize to cleave palindromic or pseudo-palindromic target sequences.

Although the LAGLIDADG peptide is the only conserved region among members of the family, these proteins share a very similar three dimensional structure. The catalytic core is flanked by two DNA-binding domains with a perfect two-fold symmetry for homodimers such as I-CreI [3], I-MsoI [4] and I-CeuI [5] and with a pseudo symmetry for monomers such as I-SceI [6], I-DmoI [7] or [8].

Each of the monomers in dimeric enzymes and both domains (for monomeric enzymes) contribute to the catalytic core, organized around divalent cations. Just above the catalytic core, the two LAGLIDADG peptides also play an essential role in the dimerization interface. DNA binding depends on two typical saddle-shaped αββαββα folds, sitting on the DNA major groove. Other domains can be found, for example in inteins such as PI-PfuI [9] and PI-SceI [10], whose protein splicing domain is also involved in DNA binding.

The making of functional chimeric meganucleases, by fusing the N-terminal I-DmoI domain with an I-CreI monomer [11-14] has demonstrated the plasticity of LAGLIDADG proteins.

Different groups have also used a semi-rational approach to locally alter the specificity of the I-CreI [15-23], I-SceI [24], PI-SceI [25] and I-MsoI [26].

In addition, hundreds of I-CreI derivatives with locally altered specificity have been engineered by combining the semi-rational approach and High Throughput Screening Method [17-21, 23, 27-30].

The combination of mutations from the two subdomains of I-CreI within the same monomer has allowed the design of novel chimeric molecules (homodimers) able to cleave new combinations of existing targets [23, 29-30].

The method for producing meganuclease variants and the assays based on cleavage-induced recombination in mammal or yeast cells, which are used for screening variants with altered specificity, are described in [13, 21 and 31-32]. These assays result in a functional LacZ reporter gene which can be monitored by standard methods.

The combination of looking for mutations in subdomains, allows a larger combinatorial approach, involving each of these substantially independent four different subdomains. The different subdomains can be modified separately and combined to obtain an entirely redesigned meganuclease variant (heterodimer or single-chain molecule) with chosen specificity. In a first step, couples of novel meganucleases are combined in new molecules (“half-meganucleases”) cleaving palindromic targets derived from the target one wants to cleave. Then, the combination of such “half-meganucleases” can result in a heterodimeric species cleaving the target of interest.

The assembly of four sets of mutations into heterodimeric endonucleases cleaving a target sequence or a sequence from different genes has been described in the following patent applications: XPC gene [33], RAG gene [34], HPRT gene [35], beta-2 microglobulin gene [36], Rosa26 gene [37], Human hemoglobin beta gene [38] and Human interleukin-2 receptor gamma chain gene [39].

These variants can be used to cleave genuine chromosomal sequences and have paved the way for novel perspectives in several fields, including gene therapy.

In order for the endonucleases to act it is necessary for them to come into contact either in vivo (namely in a cell within an intact tissue or organism) or ex vivo (namely in a cell comprised within a tissue excised and/or isolated from an organism) with the genetic material of cells comprising the genetic defect.

Viral vectors have previously been used to bring into cells the components needed for an enhanced gene targeting through HR. Viral vectors encoding within modified versions of their genome site specific endonucleases (Zinc Finger Nucleases or the I-SceI homing endonuclease) have been used to transduce cultured cells, together with vectors containing a substrate for homologous recombination called the repair matrix hereafter.

The expected 100 to 1000 fold increase in HR frequency were obtained in these proof-of-principle studies [40-43]. Viral vector based systems remain cumbersome and their disadvantages may make their use in a clinical setting difficult. For instance, three different vectors were used by Lombardo et al. [41], one for each chain of a heterodimeric nuclease of the Zinc Finger Nuclease and one for the repair matrix. The simultaneous and equal transformation of multiple viral vectors into a mixed population of cells as would be the case in even the most basic of in vivo gene therapy protocols, would be very difficult to achieve. Likewise the consolidation of all the components detailed in Lombardo et al., into a single genomic construct would also be very difficult given the inherent size constraints of the viral genome, wherein above a certain size and/or when it lacks certain key sequences the modified viral genome can no longer be packaged or contained within the virus particle.

A further disadvantage of most current viral vector systems, is that sequences encoding the endonuclease are introduced into cells as part of the genetic material of the virus and following this introduction they are transcribed and translated into the functional endonuclease. It can take several days for this process to occur and such a prolonged period of time is not be acceptable or currently possible in a clinical setting due to the cytotoxic effects of viral vector administration and problems with non-specific genomic cleavage by the endonuclease following such prolonged in vivo exposure.

As an alternative to such systems workers have also included enzymes which mediate the gene repair event in a peptide form as a component of the viral vector. One example of this (60) is the incorporation of the recombinase enzyme cre from the LoxP-cre system. The recombinase was in the form of a fusion protein with the virus protein Vpr. Such cre containing virus particles were shown to be able to induce the introduction of a target sequence present in the modified genome of the vecor at a previously introduced LoxP site in a target cell.

Such an approach however requires the alteration of the target cell line, so that it comprises the LoxP target site, prior to the introduction of the target sequence, such a two step process makes the use of such vectors impossible for routine gene therapy purposes.

The inventors seeing the disadvantages of existing means and materials to deliver meganucleases to their sites of action have developed a new set of materials which overcome the disadvantages of the prior art.

In accordance with a first aspect of the present invention there is provided a fusion protein comprising at least:

a meganuclease, which recognises and cleaves a specific DNA target sequence;

wherein said fusion protein is able to associate with virus vector particles and following transduction into a host cell recognise and cleave said specific DNA target in vivo.

The inventors have shown that functional meganuclease enzymes can be delivered to target cells in a peptide form and hence are immediately active and so can act upon a specific target in the host cell genome as soon as they are released into the cell.

Viral vectors include retrovirus, adenovirus, parvovirus (e. g. adeno-associated viruses), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomega-lovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). The term “vector” refers to a nucleic acid molecule capable of transporting another nucleic acid to which it has been linked.

The fusion protein according to the present invention is one which can associate with the components of the virus vector particles. Normally the fusion protein will consist of the meganuclease sequence fused with a peptide sequence originating from the virus which has been manipulated into a vector, examples of such proteins will in general be non-essential to vector activity. Alternatively however the fusion protein can comprise an artificial sequence which mediates the incorporation of the fusion protein into the virus vector particles, for instance a peptide sequence which forms a complex with one or more components of the virus vector particle. A final strategy is to modify a portion of the peptide sequence of the meganuclease so as to promote association with virus vector particles. Examples of possible modifications of a meganuclease (or any site specific nuclease) sequence promoting its association with a viral vector particle are fusion with the VP2 protein of the Adeno-Associated Virus [44], fusion with the Adenovirus p9 protein [45], fusion with an Herpes Simplex type 1 tegument protein such as VP16 or VP26 [46, 47], fusion with the Marek's Disease Virus protein VP22 [48], fusion with lentiviral Nef proteins [46], fusion with the Cyclosporin binding protein with interact with HIV p24gag capsid protein [49] and fusion with any peptide selected to bind HIV p6 (synthetic Vpr).

In particular there is provided a fusion protein comprising at least:

a meganuclease, which recognises and cleaves a specific DNA target sequence; and

a peptide which promotes association with virus vector particles selected from the group comprising: a virus protein or a non-virus protein, as well as a fragment or derivative thereof;

wherein said fusion protein is able to associate with virus vector particles and following transduction into a host cell recognise and cleave said specific DNA target in vivo.

In a preferred embodiment there is provided a fusion protein comprising at least:

a meganuclease, which recognises and cleaves a specific DNA target sequence;

a viral peptide selected from the group Vpr and Vpx or a fragment or derivative thereof;

wherein said fusion protein is able to associate with Lentivirus vector particles and following transduction into a host cell recognise and cleave said specific DNA target in vivo.

The inventors have experimentally validated there new system using a meganuclease::Vpr fusion protein, incorporated into lentiviral vector particles and used to transform a range of target cells at the genetic level. Such systems have previously been postulated [59] but never successfully reduced to practice using meganucleases or any site specific nuclease. That such meagnuclease fusion proteins retain their functionality is surprising given that C or N terminal extensions to meganucleases and enzymes in general have previously been shown in many cases to lead to an alteration or cessation of enzymatic activity. Such an alteration or cessation of activity would be unacceptable in the case of meganuclease fusion proteins as this would either lead to an inactive enzyme of no use or else a potentially toxic enzyme recognising and cleaving targets different to those intended. The inventors have also found that functional fusion proteins can be made using engineered variants of meganucleases, which for instance have altered target specificity or activity and also that these fusion proteins when the meganuclease component is a single chain enzyme, that is a meganuclease which is normally dimeric but which has been engineered so that the normally separate monomers are linked by an additional peptide sequence and so are present in the form of a single amino acid chain.

The inventors have shown that following transfer into the cell cytoplasm, the meganuclease retains its activity and readily accesses the nucleus where it recognizes and cleaves its target sequence.

Vpr, an “accessory” protein of HIV-1, has been shown to be essential for maintaining chronic infection in infected individuals [50] but is dispensable in the vector systems [51]. Over 200 Vpr molecules are incorporated into each virion through their interaction with the p6 Gag protein [52]. Vpx is found in HIV-2 (and SIV), but not in HIV-1. It is closely related to Vpr at the genetic level, which indicates that its existence might have come about as a duplication of the Vpr gene post divergence of these related viruses. The role of Vpx in the lifecycle of HIV is not entirely clear and it appears to be dispensable, since types of HIV-2 without a functioning Vpx gene are still able to replicate and to infect cells [53-54].

Lentiviral vectors (LV) are a class of vector derived from Lentiviruses which are a subclass of Retroviruses. LVs were developed as vectors due to the ability of the Lentiviral genome to integrate into the genome of non-dividing cells, which is a unique feature of Lentiviruses as other Retroviruses genomic materials can integrate only into the genome of dividing cells.

LVs are replication defective viral particles which comprise an inner protein core surrounding the genetic material of the virus, generally called the nucleocapsid core and an outer lipid membrane. These replication defective viral particles are assembled by expressing proteins encoded by the lentiviral gag and pol genes in packaging cells. The gag and pol genes encode polyproteins and a protease that processes these polyproteins into individual components of the virus particle.

In order to maximise the functionality of the meganuclease, the meganuclease and viral peptide can be linked by a protease cleavage site. This protease cleavage site can be a site targeted by a protease endogenous to the host cell or alternatively in a preferred embodiment this protease site is a HIV protease cleavage site and hence is acted upon by the protease associated with the Lentivirus vector particle.

In particular the DNA target sequence is from the genome of the target cell.

In accordance with this aspect of the present invention the target sequence is endogenous to the target cell genome and in particular may come from a coding or non-coding portion of a gene.

Most particularly the DNA target sequence is at or close to the site of a mutation in a gene comprised with the target cell genome that is associated with a genetic disease.

In particular the fusion protein comprises positioned between the meganuclease and the viral peptide, a protease cleavage site.

In particular the HIV protease cleavage site is selected from the group 7/1 (SEQ ID NO: 1) and 24/2 (SEQ ID NO: 2).

In particular the fusion protein comprises a detectable tag.

The incorporation of a detectable tag, allows the presence and concentration of the tagged protein to be determined in vivo.

In particular the detectable tag is attached to the NH₂ and/or COOH terminus of the meganuclease peptide and/or positioned upon the NH₂ and/or COOH terminus and/or within the fusion protein.

Several detectable tags are known in the art such as HIS-HHHHHH—(SEQ ID NO: 3), c-MYC-EQKLISEEDL—(SEQ ID NO: 4), HA YPYDVPDYA—(SEQ ID NO: 5) VSV-G YTDIEMNRLGK—(SEQ ID NO: 6), HSV-QPELAPEDPED—(SEQ ID NO: 7), V5-GKPIPNPLLGLDST—(SEQ ID NO: 8), FLAG-DYKDDDDK—(SEQ ID NO: 9).

In particular the fusion protein is characterized in that it comprises a Nuclear Localisation Signal (NLS) at the NH₂ and/or COOH terminus or comprised with the fusion protein. Most particularly the NLS is located at the NH₂ and/or COOH terminus of the meganuclease peptide sequence.

A NLS is an amino acid sequence which acts to target the protein to the cell nucleus through the Nuclear Pore Complex and to direct a newly synthesized protein into the nucleus via its recognition by cytosolic nuclear transport receptors. Typically, a NLS consists of one or more short sequences of positively charged amino acids such as lysines or arginines.

In particular the NLS is selected from the NLS sequences of the known proteins SV40 large T antigen-PKKKRKV—(SEQ ID NO: 10), nucleoplasmin-KR[PAATKKAGQA]KKKK—(SEQ ID NO: 11), p54-RIRKKLR—(SEQ ID NO: 12), SOX9-PRRRK—(SEQ ID NO: 13), NS5A-PPRKKRTVV—(SEQ ID NO: 14).

In particular the meganuclease which forms a part of the fusion protein, is selected from the group comprising: I-SceI, I-ChuI, I-Cre I, I-DmoI, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, I-MsoI, PI-Tsp I; and variants or derivatives thereof.

The inventors and others have isolated, characterised and further developed a large range of meganucleases and variants thereof which target a large number of gene loci such as XPC gene [33], RAG gene [34], HPRT gene [35], beta-2 microglobulin gene [36], Rosa26 gene [37], Human hemoglobin beta gene [54] and Human interleukin-2 receptor gamma chain gene [39]. Any and all of such meganucleases and further materials derived there from can be used in the present invention.

For meganucleases which act as dimers for instance I-CreI, wherein this meganuclease acts as a homodimer with two identical fusion proteins which can come together so as to form the required dimer. Wherein the enzyme acts as a heterodimer, it is necessary to generate two fusion proteins which differ as to the peptide sequence of the meganuclease they comprise. Both of these fusion proteins can then be incorporated in the Lentivirus vector and following host cell entry can form the required heterodimeric enzyme. An alternative is to produce a single chain derivative of the two monomers joined by a peptide linker and form a fusion protein using this.

In particular the viral peptide consists of SEQ ID NO: 15.

The inventors have found that a fragment of the HIV1 Vpr protein, corresponding to residues 1 to 96 of this protein, is particularly suited to fusion with a meganuclease in accordance with the present invention. Alternatively a peptide from position 14 to 88 of Vpr (SEQ ID NO: 16) may be used as well. The Vpr moiety may be placed either in NH₂ and/or COOH terminus of the fusion protein.

Alternatively the viral peptide may be the Vpr protein from HIV2 (SEQ ID NO: 17) or the Vpx protein (SEQ ID NO: 18) from the Simian Immunodeficiency virus (SIV) as well as fragments and derivatives thereof.

According to a second aspect of the present invention there is provided a polynucleotide, which encodes a fusion protein according to the first aspect of the present invention.

According to a third aspect of the present invention there is provided a lentiviral vector particle comprising at least one fusion protein according to the first aspect of the present invention.

In particular the lentiviral vector, further comprises a DNA molecule encoding a repair matrix (RMA).

The RMA is a DNA construct comprising a first and second portions which are homologous to regions 5′ and 3′ of the DNA target in situ. Following cleavage of the DNA target, a homologous recombination event is stimulated between the genome and the RMA, wherein the genomic sequence containing the DNA target is replaced by the third portion of the RMA and a variable part of the first and second portions of the RMA.

The inventors have demonstrated that the same lentiviral particle can be used to deliver a nucleic acid sequence for homologous recombination at the chromosomal locus of meganuclease cleavage as well as meganuclease containing fusion proteins.

In this preferred embodiment of the present invention all the components of the gene targeting system are comprised within a single entity, the Lentiviral vector particle. Therefore using such a unified construct it is only necessary to optimize the administration conditions for this single transformative entity and not as for prior art approaches the optimization of administration conditions for several vectors.

The RMA may be present as a single or multiple copies per Lentivirus vector particle and in particular may be integrated into the modified viral genome or present as an episomal DNA molecule.

In accordance with a further aspect of the present invention there is provided a viral vector which comprises:

a fusion protein which comprises an enzyme able to alter the DNA content of a host cell, wherein said fusion protein is able to associate with the viral vector particles; and

a DNA molecule which the enzyme can use to alter the DNA content of the host cell;

wherein following transduction of the viral vector particle into a host cell, the enzyme can recognise and alter the DNA content of the host cell in vivo.

Besides the delivery of meganucleases, the viral and in particular lentiviral vector system described herein could be used to transfer other types of natural and engineered endonucleases, recombinases or transposases, together with a DNA substrate for recombination or transposition. For example, heterodimeric meganucleases or Zinc finger nucleases could be incorporated into virions by fusing their two constitutive chains and separating them by a lentiviral protease cleavage site. Transposases such as the one encoded by the Sleeping Beauty transposon [55] or integrases like in the PhiC31 phage [56] may also be incorporated for efficient, nucleic acid-free transfer into vertebrate cells.

In particular the lentiviral vector lacks functional viral integrase.

The integration of the virus vector genome into the host cell genome can lead to insertional mutagenesis and these mutations can have a deleterious effect upon the host cell. As it is not necessary or desirable for the viral genome and/or in particular the RMA to integrate into the host cell genome the gene product which mediates genomic integration, the viral integrase.

In particular the lentiviral vector comprises exogenous surface antigens.

Heterologous transmembrane glycoproteins such as the Surface Unit (SU) proteins from gamma retroviruses or the G protein from the Vesicular Stomatitis Virus (VSV) can also be expressed in LV packaging systems and are there after incorporated into the lipid membrane surrounding the nucleocapsid core. These lipid membrane bound proteins mediate the first step of the entry process following recognition of a receptor molecule on the surface of target cells.

According to a fourth aspect of the use of a fusion protein according to the first aspect of the present invention or a polynucleotide according to the second aspect of the present invention or a lentiviral vector particle according to the third aspect of the present invention, to alter a genomic DNA sequence present in a target cell in vitro.

According to a fifth aspect of the present invention there is provided a medicament comprising at least a fusion protein according to the first aspect of the present invention or a polynucleotide according to the second aspect of the present invention or a lentiviral vector particle according to the third aspect of the present invention.

In particular the materials according to the present invention may be used in a gene therapy method so as to correct, alter or supplement one or more genetic defects in a cell, cell population, tissue or organism either via the treatment of an isolated material or on a whole organism basis. The meganucleases which have been previously isolated for the purposes of treating a genetic disease are particularly useful according to this aspect of the present invention when formed into fusion proteins as per the current invention.

In addition to the treatment by gene therapy of a disease the materials and methods disclosed in the present Patent Application can also be used to research aspects of genetic disease, gene function or more fundamental aspects of biology such as development, by altering gene expression levels/gene product activity via an alteration to a gene coding sequence or regulatory sequence using the materials and methods described herein.

According to a sixth aspect of the present invention there is provided a host cell, characterized in that it is modified by a fusion protein a fusion protein according to the first aspect of the present invention or a polynucleotide according to the second aspect of the present invention or a lentiviral vector particle according to the third aspect of the present invention.

According to a seventh aspect of the present invention there is provided a non-human transgenic animal, characterized in that all or part of its cells have been modified by a fusion protein according to the first aspect of the present invention or a polynucleotide according to the second aspect of the present invention or a lentiviral vector particle according to the third aspect of the present invention.

According to an eighth aspect of the present invention there is provided a transgenic plant or tissue thereof, characterized in that all or part of its cells have been modified by a fusion protein according to the first aspect of the present invention or a polynucleotide according to the second aspect of the present invention or a vector particle comprising a fusion protein according to the first aspect of the present invention.

Definitions

Throughout the present Patent Application a number of terms and features are used to present and describe the present invention, to clarify the meaning of these terms a number of definitions are set out below and wherein a feature or term is not otherwise specifically defined or obvious from its context the following definitions apply.

Amino acid residues in a polypeptide sequence are designated herein according to the one-letter code, in which, for example, Q means Gln or Glutamine residue, R means Arg or Arginine residue and D means Asp or Aspartic acid residue.

Amino acid substitution means the replacement of one amino acid residue with another, for instance the replacement of an Arginine residue with a Glutamine residue in a peptide sequence is an amino acid substitution.

by “altered specificity” it is intended a meganuclease variant, derivative or fragment which recognizes and cleaves a DNA target different to the target of a parent or original meganuclease from which it is derived.

by “associate with a virus vector particle” it is intended to mean the property of a fusion protein according to the present invention to associate with the other components of a virus vector particle either in vivo or in vitro such that when the virus particles are generated the fusion protein is incorporated therein.

by “chimeric DNA target” or “hybrid DNA target” it is intended the fusion of a different half of two parent meganuclease target sequences. In addition at least one half of said target may comprise the combination of nucleotides which are bound by at least two separate subdomains (combined DNA target).

by “chimeric meganuclease” it is intended to mean a meganuclease which comprises functional portions of at least two different meganucleases or variants of the same meganuclease and which can recognise and cleave a DNA target sequence.

Cleavage activity: the cleavage activity of a variant, derivative or fragment of a meganuclease according to the invention may be measured by any well-known, in vitro or in vivo cleavage assay, such as those described in the International PCT Application WO 2004/067736; Epinat et al., Nucleic Acids Res., 2003, 31, 2952-2962; Chames et al., Nucleic Acids Res., 2005, 33, e178; Arnould et al., J. Mol. Biol., 2006, 355, 443-458, and Arnould et al., J. Mol. Biol., 2007, 371, 49-65. For example, the cleavage activity of a meganuclease of the invention may be measured by a direct repeat recombination assay, in yeast or mammalian cells, using a reporter vector. The reporter vector comprises two truncated, non-functional copies of a reporter gene (direct repeats) and the genomic (non-palindromic) DNA target sequence within the intervening sequence, cloned in a yeast or a mammalian expression vector.

by “selection or selecting” it is intended to mean the isolation of one or more meganuclease variants based upon an observed specified phenotype, for instance altered cleavage activity. This selection can be of the variant in a peptide form upon which the observation is made or alternatively the selection can be of a nucleotide coding for selected meganuclease variant.

by “screening” it is intended to mean the sequential or simultaneous selection of one or more meganuclease variant (s) which exhibits a specified phenotype such as altered cleavage activity.

by “derived from” it is intended to mean a meganuclease variant which is created from a parent meganuclease and hence the peptide sequence of the meganuclease variant is related to (primary sequence level) but derived from (mutations) the sequence peptide sequence of the parent meganuclease.

by “derivative” it is intended to mean a portion of a molecule for instance a peptide derived from a protein, which shares some structural or functional features with the protein but is not identical to it. In particular a derivative may comprise an additional sequence such as a detectable tag or alternatively may be a portion of the protein which lacks some sections of the protein but retains a desired property or feature such as the ability to associate with a viral vector particle or in the case of a meganuclease derivative the ability to recognise and cleave a specific DNA target.

by “domain” or “core domain” it is intended the “LAGLIDADG homing endonuclease core domain” which is the characteristic α₁β₁β₂α₂β₃β₄α₃ fold of the homing endonucleases of the LAGLIDADG family, corresponding to a sequence of about one hundred amino acid residues. Said domain comprises four beta-strands (β₁β₂β₃β₄) folded in an antiparallel beta-sheet which interacts with one half of the DNA target. This domain is able to associate with another LAGLIDADG homing endonuclease core domain which interacts with the other half of the DNA target to form a functional endonuclease able to cleave said DNA target. For example, in the case of the dimeric homing endonuclease I-CreI (163 amino acids), the LAGLIDADG homing endonuclease core domain corresponds to the residues 6 to 94.

by “DNA target”, “DNA target sequence”, “target sequence”, “target-site”, “target”, “site”; “site of interest”; “recognition site”, “recognition sequence”, “homing recognition site”, “homing site”, “cleavage site” it is intended a 20 to 24 by double-stranded palindromic, partially palindromic (pseudo-palindromic) or non-palindromic polynucleotide sequence that is recognized and cleaved by a LAGLIDADG homing endonuclease such as I-CreI, or a variant, or a single-chain chimeric meganuclease derived from I-CreI. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicated for C1221 (SEQ ID NO: 19). Cleavage of the DNA target occurs at the nucleotides at positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.

by “DNA target half-site”, “half cleavage site” or half-site” it is intended the portion of the DNA target which is bound by each LAGLIDADG homing endonuclease core domain.

by “fragment” it is intended to mean a derivative which comprises only a portion of an original protein, but which retains a desired property such the ability to associate with a viral vector particle or cleave a specific target. The fragment may in addition also comprise additional sequences such as a detectable tag or other component.

by “functional variant” it is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.

by “heterodimer” it is intended to mean a meganuclease comprising two non-identical monomers. In particular the monomers may differ from each other in their peptide sequence and/or in the DNA target half-site which they recognise and cleave.

by “homologous” is intended a sequence with enough identity to another one to lead to a homologous recombination between sequences, more particularly having at least 95% identity, preferably 97% identity and more preferably 99%.

by “I-CreI” it is intended the wild-type I-CreI having the sequence of pdb accession code 1 g9y, corresponding to the sequence SEQ ID NO: 20 in the sequence listing. In the present Patent Application the I-CreI variants described comprise an additional Alanine after the first Methionine of the wild type I-CreI sequence. These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present Patent Application, as these references exclusively refer to residues of the wild type I-CreI enzyme (SEQ ID NO: 20) as present in the variant, so for instance residue 2 of I-CreI is in fact residue 3 of a variant which comprises an additional Alanine after the first Methionine.

by “I-CreI site” it is intended a 22 to 24 by double-stranded DNA sequence which is cleaved by I-CreI. I-CreI sites include the wild-type (natural) non-palindromic I-CreI homing site and the derived palindromic sequences such as the sequence 5′-t₋₁₂c₋₁₁a₋₁₀a₋₉a−8a_{31 7}c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₊₁₂ (SEQ ID NO: 19), also called C1221.

“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.

by “meganuclease”, it is intended an endonuclease having a double-stranded DNA target sequence of 12 to 45 bp. The meganuclease is either a dimeric enzyme, wherein each domain is on a monomer or a monomeric enzyme comprising the two domains on a single polypeptide.

by “meganuclease domain”, it is intended the region which interacts with one half of the DNA target of a meganuclease and is able to associate with the other domain of the same meganuclease which interacts with the other half of the DNA target to form a functional meganuclease able to cleave said DNA target.

by “meganuclease variant” or “variant” it is intended a meganuclease obtained by replacement of at least one residue in the amino acid sequence of the parent meganuclease (natural or variant meganuclease) with a different amino acid.

by “monomer” it is intended to mean a peptide encoded by the open reading frame of a dimeric meganuclease gene or a variant thereof, which when allowed to dimerise forms a functional meganuclease enzyme. In particular the monomers dimerise via interactions mediated by the LAGLIDADG motif.

by “monomeric” it is intended to mean a meganuclease enzyme which acts as a monomer.

by “mutation” is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.

Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.

by “peptide linker” it is intended to mean a peptide sequence of at least 10 and preferably at least 17 amino acids which links the C-terminal amino acid residue of the first monomer to the N-terminal residue of the second monomer and which allows the two variant monomers to adopt the correct conformation for activity and which does not alter the specificity of either of the monomers for their targets.

by “pi-10” or “π-10” it is intended to mean a genetic construct present in the genome of a cell, comprising amongst other components, a meganuclease cleavage site flanked by two regions which are homologous to two regions present in the targeting DNA construct/minimal repair matrix/repair matrix (defined below). Following cleavage of the meganuclease cleavage site a homologous recombination event between the homologous portions of the π-10 site in the host cell genome and the portions of the targeting DNA construct results in the insertion into the target cell genome of any sequence disposed in the target DNA construct between two homologous portions present therein. The further designation CHOpi-10 or CHO-π10 refers to a CHO cell line comprising the genetic construct; similarly HEK293 Pi-10 refers to a HEK293 cell line comprising the genetic construct, etc.

by “subdomain” it is intended the region of a LAGLIDADG homing endonuclease core domain which interacts with a distinct part of a homing endonuclease DNA target half-site.

by “single-chain meganuclease”, “single-chain chimeric meganuclease”, “single-chain meganuclease derivative”, “single-chain chimeric meganuclease derivative” or “single-chain derivative” it is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.

by “targeting DNA construct/minimal repair matrix/repair matrix” it is intended to mean a DNA construct comprising a first and second portions which are homologous to regions 5′ and 3′ of the DNA target in situ. The DNA construct also comprises a third portion positioned between the first and second portion which comprise some homology with the corresponding DNA sequence in situ or alternatively comprise no homology with the regions 5′ and 3′ of the DNA target in situ. Following cleavage of the DNA target, a homologous recombination event is stimulated between the genome and the repair matrix, wherein the genomic sequence containing the DNA target is replaced by the third portion of the repair matrix and a variable part of the first and second portions of the repair matrix.

by “vector” is intended a nucleic acid molecule capable of transporting another nucleic acid to which it has been linked into a host cell in vitro, in vivo or ex vivo.

by “viral vector” it is intended a modified virus particle which can be used to introduce a nucleic acid molecule and/or a peptide or other molecule into a target cell.

For a better understanding of the invention and to show how the same may be carried into effect, there will now be shown by way of example only, specific embodiments, methods and processes according to the present invention with reference to the accompanying drawings in which:

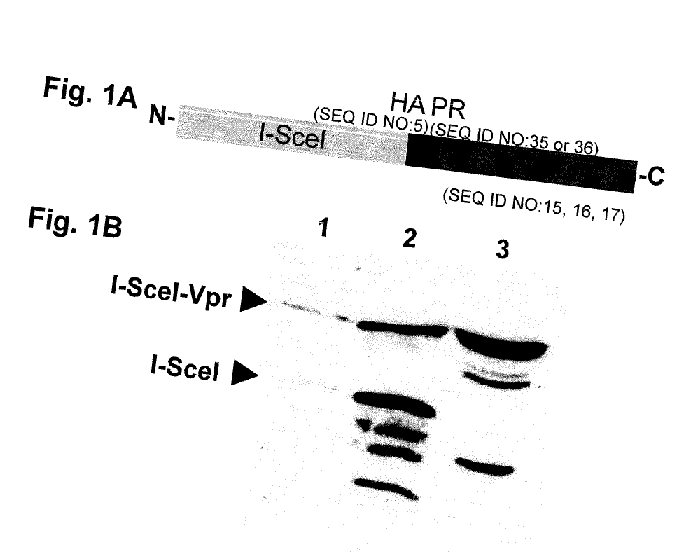

FIG. 1 shows A. a schematic representation of the structure of the I-SceI-Vpr fusion protein, in which the label HA corresponds to a Haemagglutinin tag and PR corresponds to a protease cleavage site which can be either the 7/1 or 24/2 version of the HIV protease cleavage site. B. A western blot analysis of transfected cells and purified virions using an anti-HA antibody. Lane 1—ψ1I-SceI(RMA) particles; Lane 2—ψ2I-SceI(RMA) particles; Lane 3—Cell extract from 293 cells transfected with the I-SceI::Vpr2 fusion construct.

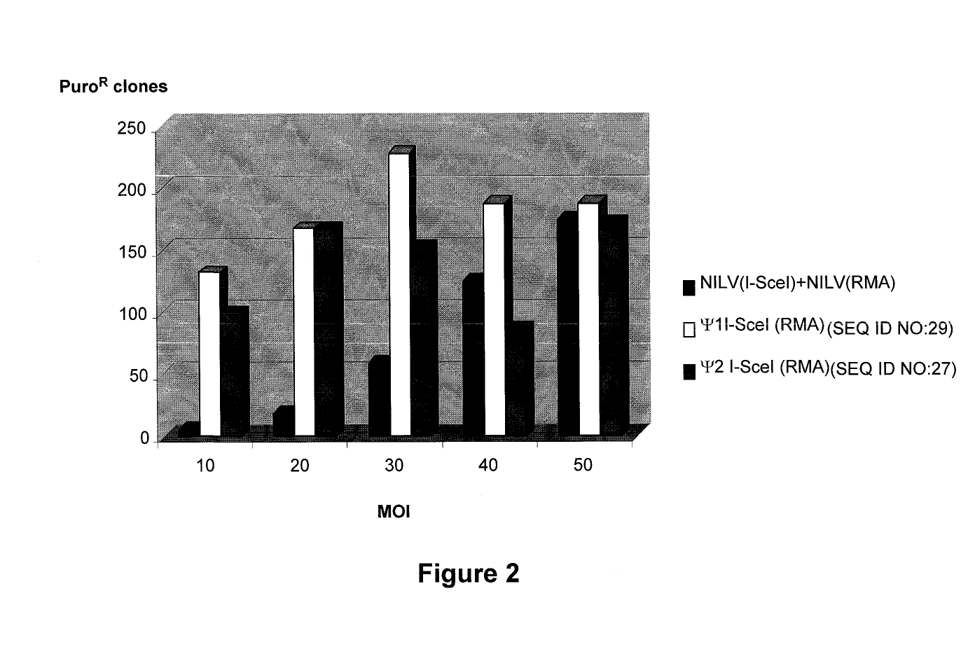

FIG. 2 shows the frequency of homologous recombination in CHO Pi-10 cells following transfer of virion-associated I-SceI. The RMA was co-packaged protein fusion ψ1I-SceI(RMA) column 2 and ψ2I-SceI(RMA) column 3. Vectors were used at increasing multiplicities of infection (moi). Controls including an Non integrative lentiviral vector (NILV) encoding I-SceI are shown in the first bar of each chart.

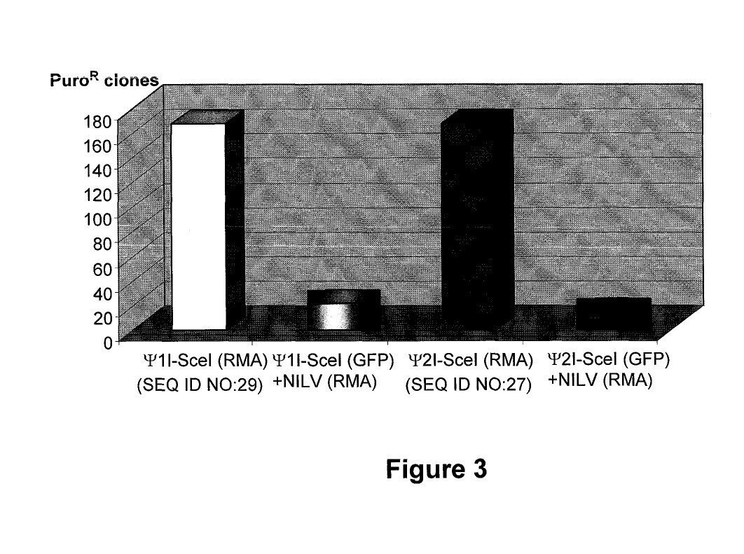

FIG. 3 shows the frequency of homologous recombination in CHO Pi-10 cells following the transfer of virion-associated I-SceI. The repair matrix (RMA) was either co-packaged (ψI-SceI(RMA)) or packaged in a separate vector (NIVL(RMA). Vectors were used at a multiplicity of infection of 20.

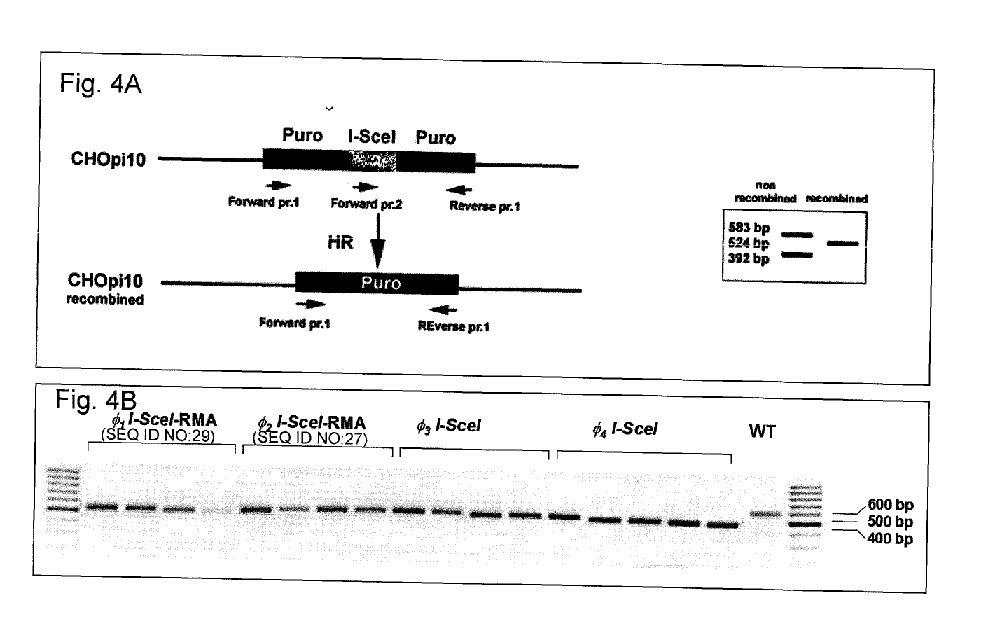

FIG. 4 shows A. schematic representations of the Puro transgene locus and reconstituted Puro locus following a homologous recombination event. B. PCR analysis of puromycin resistant clones. Recombined clones were treated with different viral preparations containing a Vpr fused I-SceI. The Repair Matrix was delivered in cis for ψ1I-SceI(RMA), ψ2I-SceI(RMA) or in trans for ψ3I-SceI and ψ4I-SceI. A 100 bp ladder was used to size the fragment and the DNA of untreated CHO pi-10 cells was used as WT control.

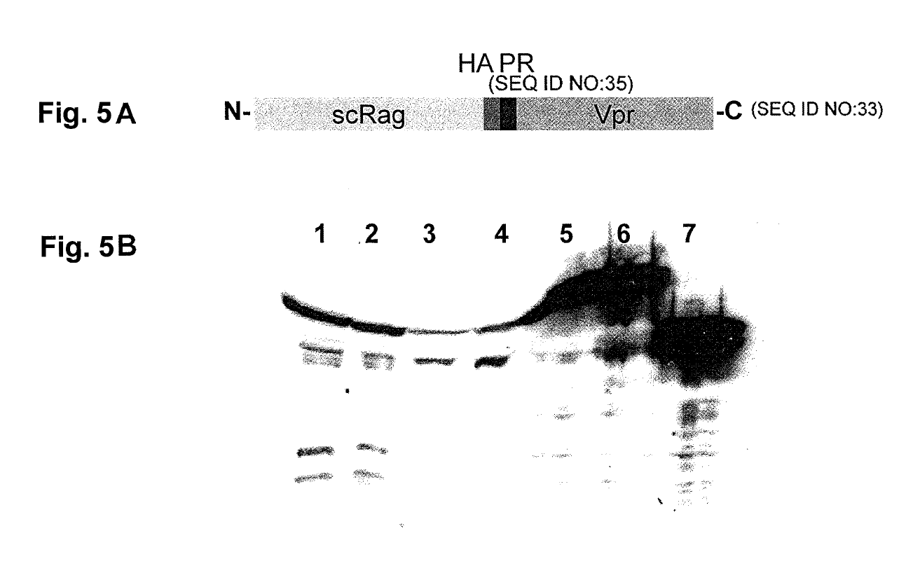

FIG. 5 shows A. a schematic representation of the structure of the fusion protein scRag::Vpr, in which the label HA corresponds to a Haemagglutinin tag and wherein the PR is the 7/1 cleavage site of the HIV protease. B. shows a western blot analysis of transfected cells and purified virions using an anti-HA antibody, in which Lane 1—ψ0scRag(GFP) particles; Lane 2—ψ0scRag(RMA) particles; Lane 3—ψ1scRag(GFP); Lane 4—ψ1scRag(RMA).

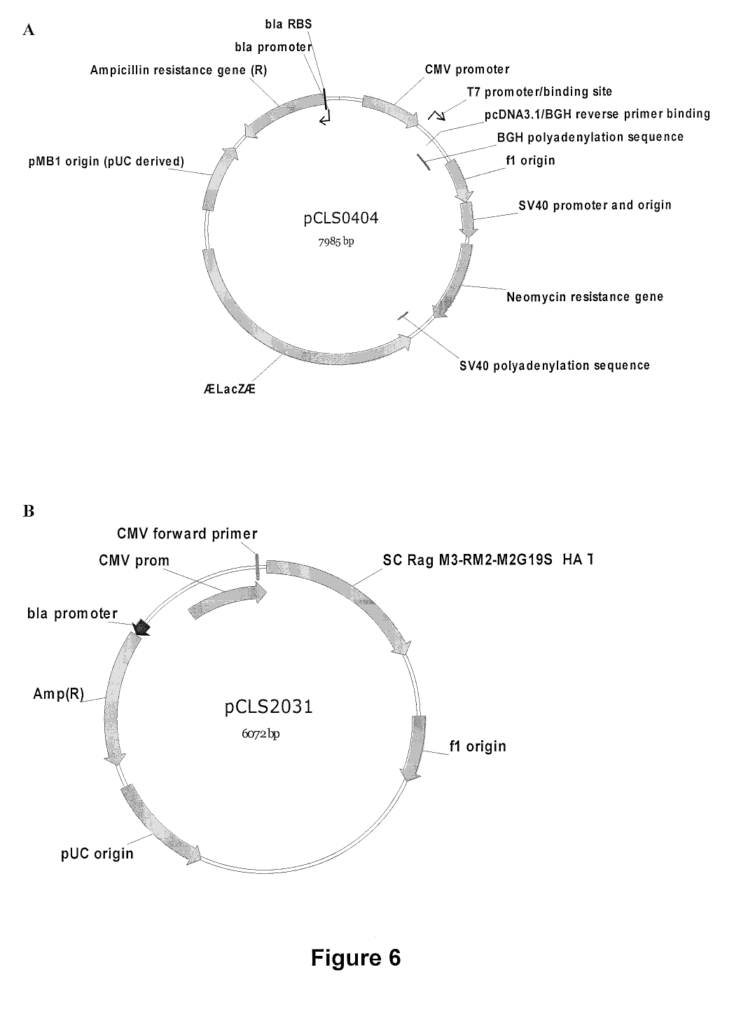

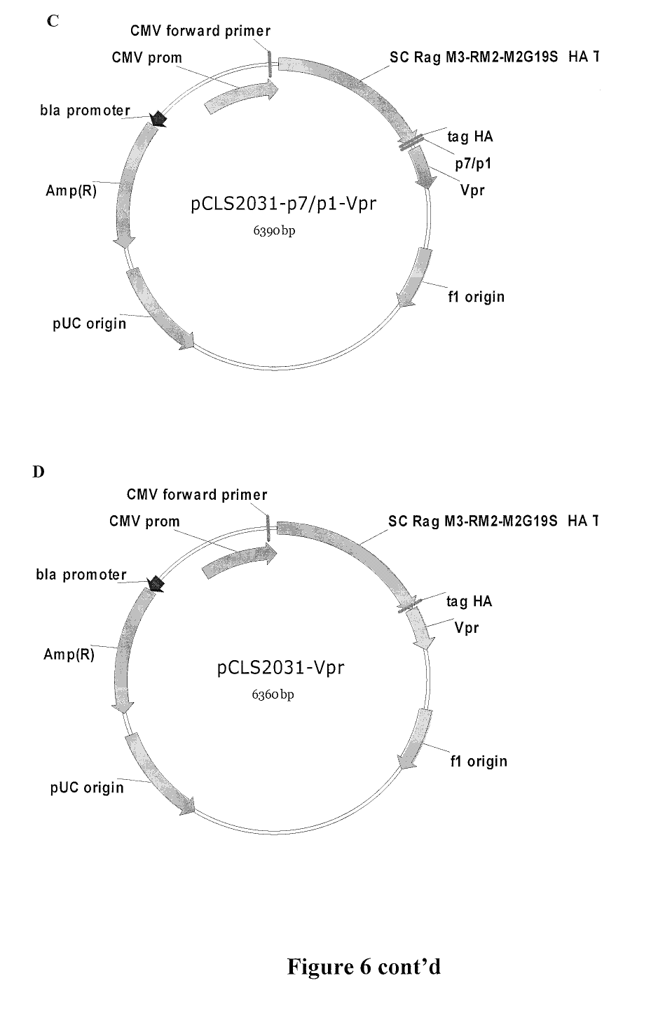

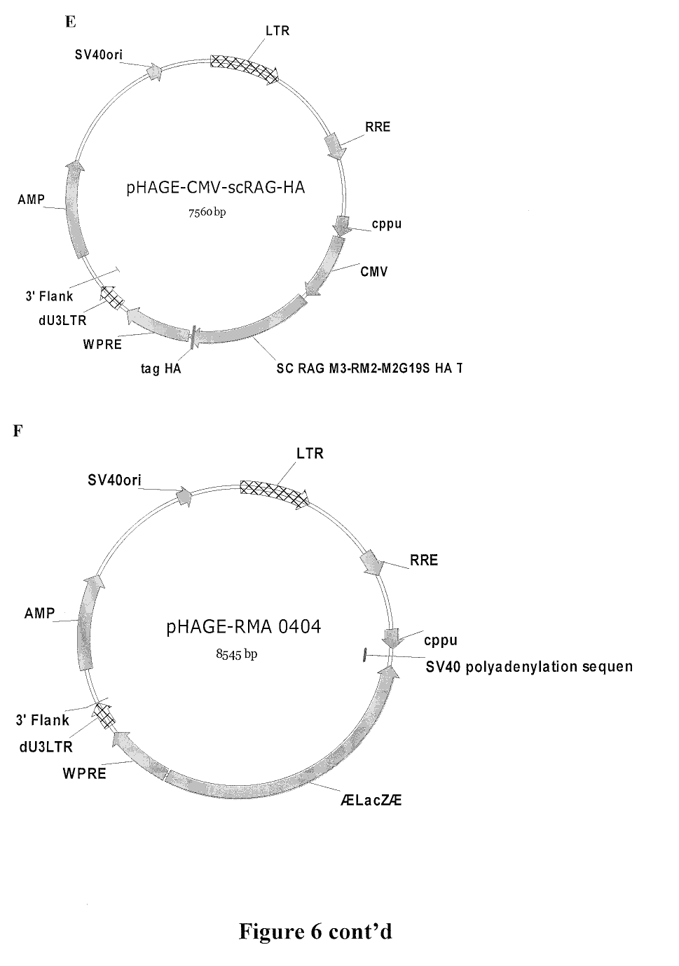

FIG. 6 shows a plasmid map of A. pCLS0404; B. pCLS2031; C. pCLS2031-p7/p1-Vpr; D. pCLS2031-Vpr; E. pHAGE-CMV-scRAG-HA; F. pHAGE-RMA 0404.

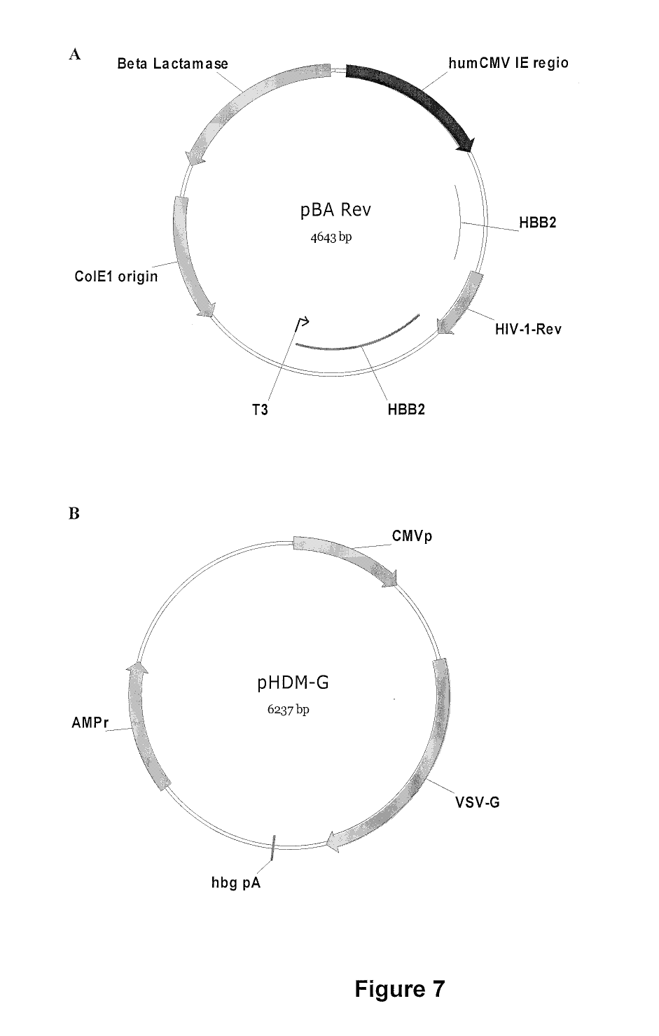

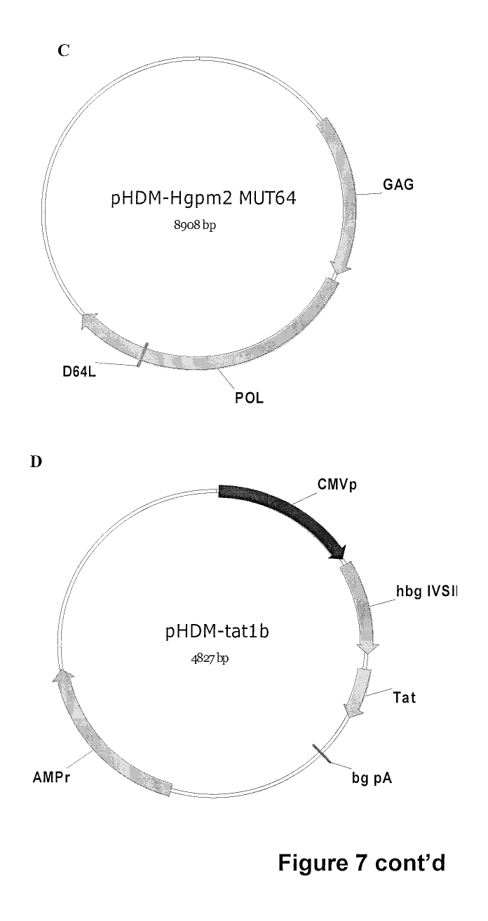

FIG. 7 shows a plasmid map of A. pBA Rev; B. pHDM-G; C. pHDM-Hgpm2 MUT64; D. pHDM-tat1b.

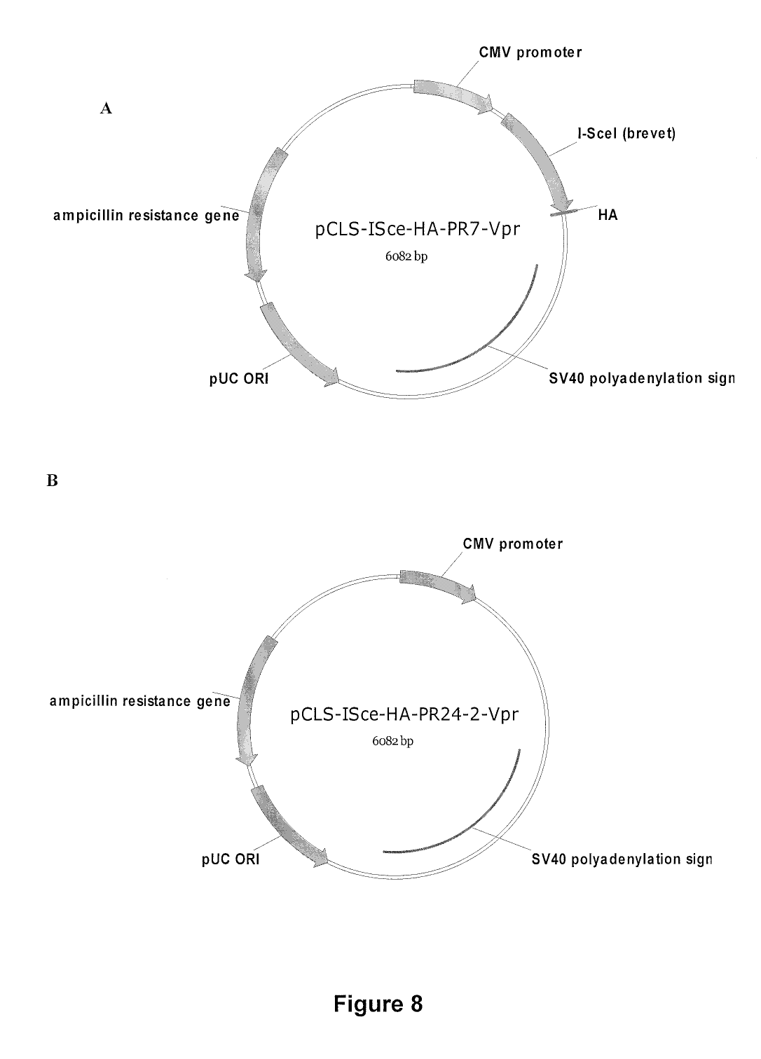

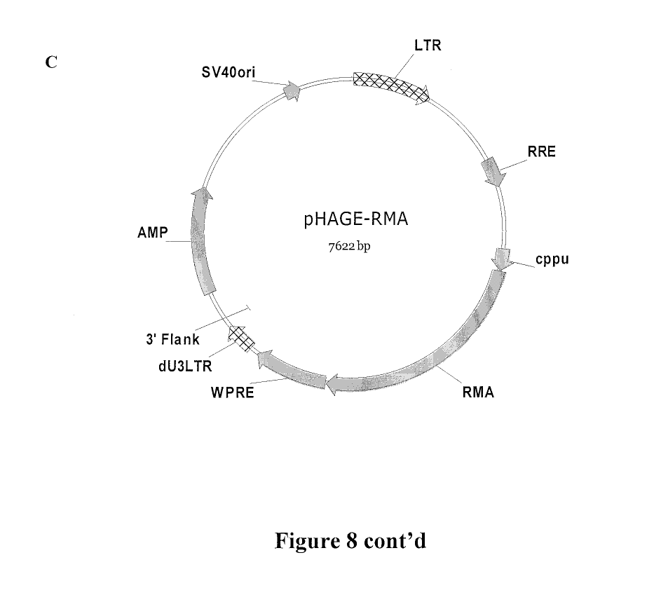

FIG. 8 shows a plasmid map of A. pCLS-ISce-HA-PR7-Vpr; B. pCLS-ISce-HA-PR24-2-Vpr; C.pHAGE-RMA.

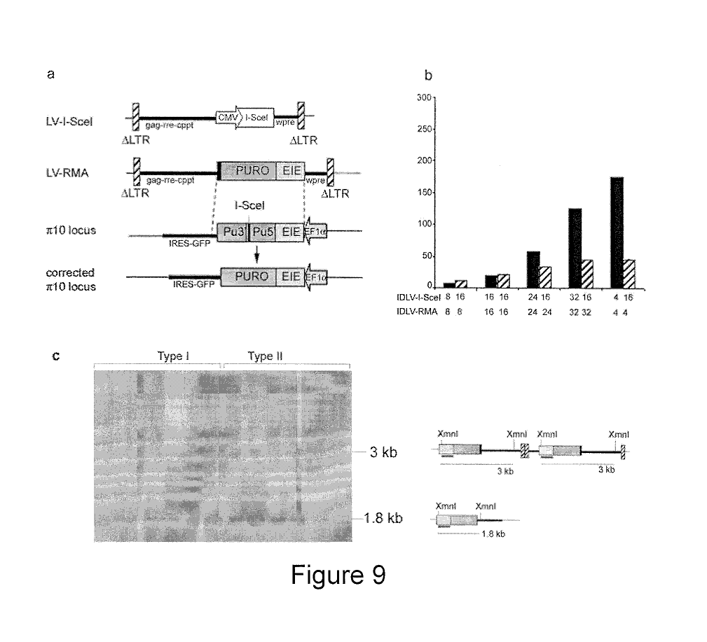

FIG. 9. Lentiviral vectors for π10 locus targeting. (a) Top: schematic of lentiviral vectors encoding the I-SceI meganuclease (LV-I-SceI) and the recombination matrix (LV-RMA). The RMA contains donor sequences homologous to exon1-intron1-exon2 (EIE) sequences of the EF1α (Elongation factor 1α) gene, a puromycin resistance gene (Puro) and part of the internal ribosomal entry site (IRES) from EMCV. LTR, long terminal repeat; wpre, woodchuck hepatitis virus post-transcription regulatory element, rre, rev responsive element; cppt, central polypurine tract. Middle: Organisation of the π10 locus including the promoter and EIE sequences from the EF1α gene driving a defective Puromycin resistance gene followed by an IRES-GFP cassette. The Puro marker is interrupted by a 55 by I-SceI recognition sequence. Bottom: Structure of the π10 locus after homologous recombination with the RMA. (b) 10e5 CHOπ10 cells were treated with the indicated doses of IDLV (pg HIV-1 p24Gag/cell). 72 hours after transduction, cells were treated with puromycin during 15 days. PuroR+ clones were counted. Data shown are representative for three independent experiments wherein a dose response when the cells were treated with increasing doses of IDLVs, but a low number of PuroR+ clones when I-SceI dose is limited to 0.16 pg HIV-1 Gag p24/cell. No PuroR+ clones were obtained in the absence of I-SceI. (c) Genomic DNAs from the Puromycin resistant clones were digested with XmnI and I-SceI and analysed by Southern blot with a ³²P-labeled internal EIE probe (underlined on the right). Fragment sizes for: Type I—correctly recombined clones, 1.8 kb; Type II—recombined clones with concatemerized RMA, 3 kb. The corresponding type I and Type II structures are drawn on the right.

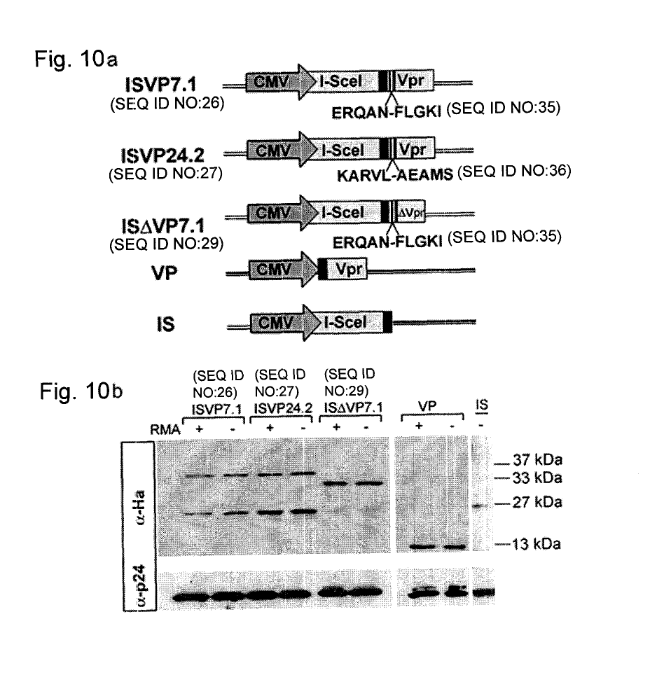

FIG. 10. Packaging I-SceI into lentiviral particles. (a) I-SceI was fused to the N terminus of full-length (ISVP7.1, ISVP24.2) or truncated (ISΔVP7.1) Vpr. I-SceI was HA tagged (black box) and different viral Gag protein cleavage sites (p7/1, p24/2) were introduced upstream of Vpr (stripped box). These fused proteins were used for I-SceI packaging with or without a viral genome encoding RMA in the same particle. VP and IS are Ha tagged Vpr or I-SceI expression cassettes used as control. (b) Western blot analysis of I-SceI containing particles. Viral particles (45-50 ng HIV-1 p24Gag) were loaded on a 10% SDS PAGE gel and probed with an antibody against the HA tag. Two bands corresponding to the cleaved (27 kDa) and non cleaved (37 or 33 kDa) proteins were obtained for ISVP7.1, ISVP24.2 and ISΔVP7.1. Note less incorporation and cleavage efficiency for the ISΔVP7.1 construct. The expected 13 kDa band was obtained using the VP control. The IS control lane contains a 27 kDa band indicating a background level (less than 10%) of I-SceI packaging in the absence of Vpr. The same blot was used for an antibody against HIV-1 Gag p24.

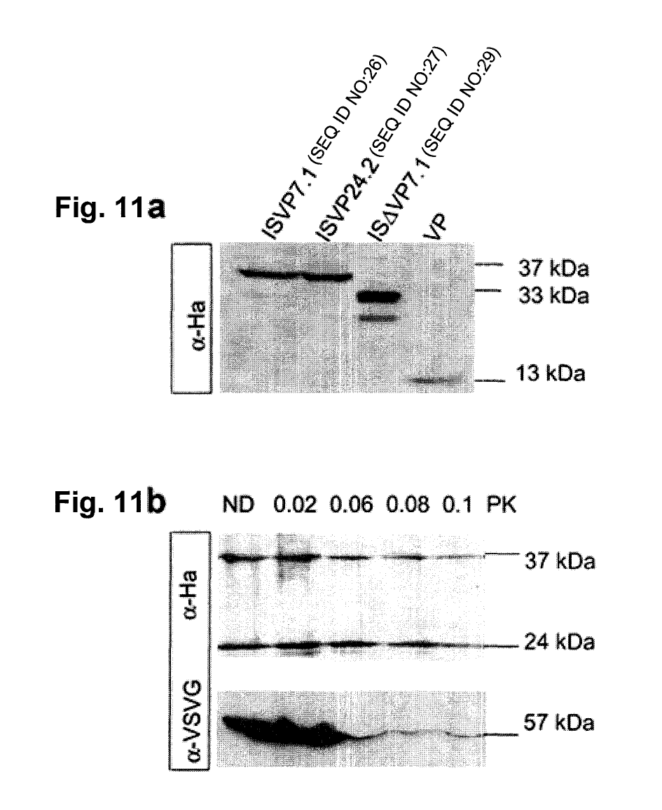

FIG. 11. Characterization of I-SceI—Vpr fusions. (a) Western blot analysis of I-SceI-Vpr fusion constructs were transfected into 293 T cells. Cells were harvested after 32 hours, lysates were loaded on 10% SDS PAGE gels and probed with an antibody against the HA tag. The expected bands corresponding to ISVP7.1 (37 kDa), ISVP24.2 (37 kDa), ISΔVP7.1 (33 kDa) and VP (15 kDa) are detected. (b) I-SceI incorporation analysis by Proteinase K digestion. ISVP24.2 particles (50 ng p24) were treated with the indicated doses of PK (mg/ml), loaded on 12% SDS PAGE gel and probed with an antibody against the HA tag. PK The same blot was probed with an antibody against VSV-G. Less VSV-G protein was detected when I-SceI containing particles were treated with 0.06 mg/ml PK, whereas the ISVP24.2 protein was still present.

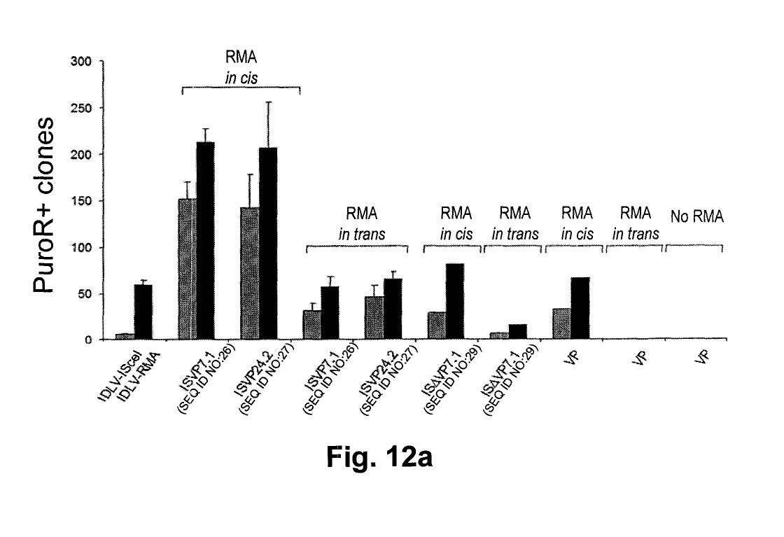

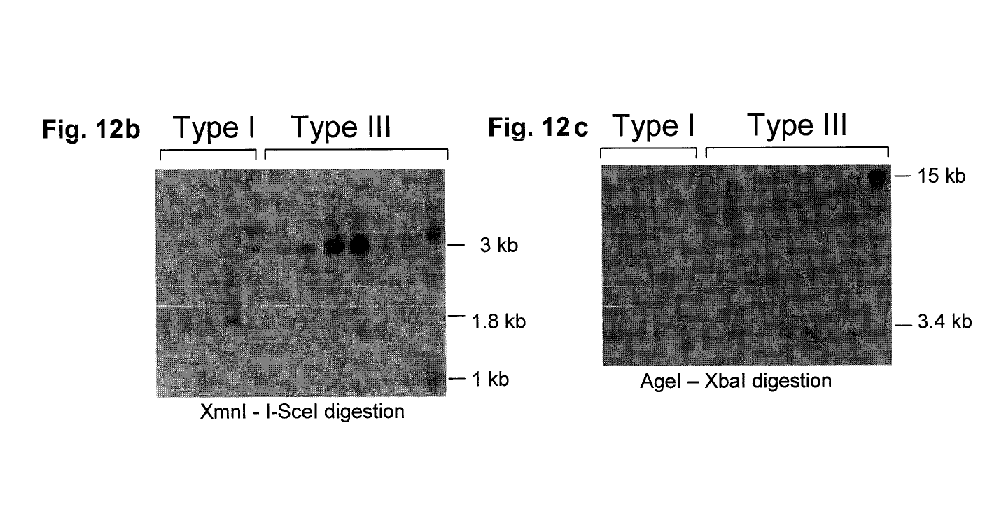

FIG. 12. π10 locus targeting with lentiviral particles containing I-SceI. (a) 10e5 reporter cells were treated with IDLVs prepared in the presence of ISVP7.1, ISVP24.2, ISΔVP7.1, VP or IS protein expression constructs. The RMA was brought either in cis or in trans. LV-I-SceI that encodes I-SceI (see FIG. 9) was used as a control. 72 hours after transduction, cells were treated with puromycin during 15 days and PuroR+ clones were counted. The data shown are representative for three independent experiments. The highest number of PuroR+clones was obtained when I-SceI containing particles were used and RMA was brought in cis. Fivefold less PuroR+ clones were obtained when the RMA was brought in trans or when a truncated Vpr was used. Few PuroR+ clones were obtained with VP particles when the RMA was brought in cis, and none when it was brought in trans. (b) Southern blot analysis of PuroR+ clones. Genomic DNA from the randomly isolated and amplified PuroR+ clones was double digested with XmnI and I-SceI and hybridized with a ³²P-labeled EIE probe. Calculated fragment sizes for: wt, 1 kb; Type I clones, 1.8 kb; Type III clones, 1 kb+3 kb (+8 kb, with tail to tail concatemers). (c) The same genomic DNAs were digested with AgeI and XbaI and hybridized with the ³²P-labeled internal EIE probe. Expected fragment sizes: Type I clones, 3.4 kb; Type III clones 3.4 kb+a band of >8.0 kb.

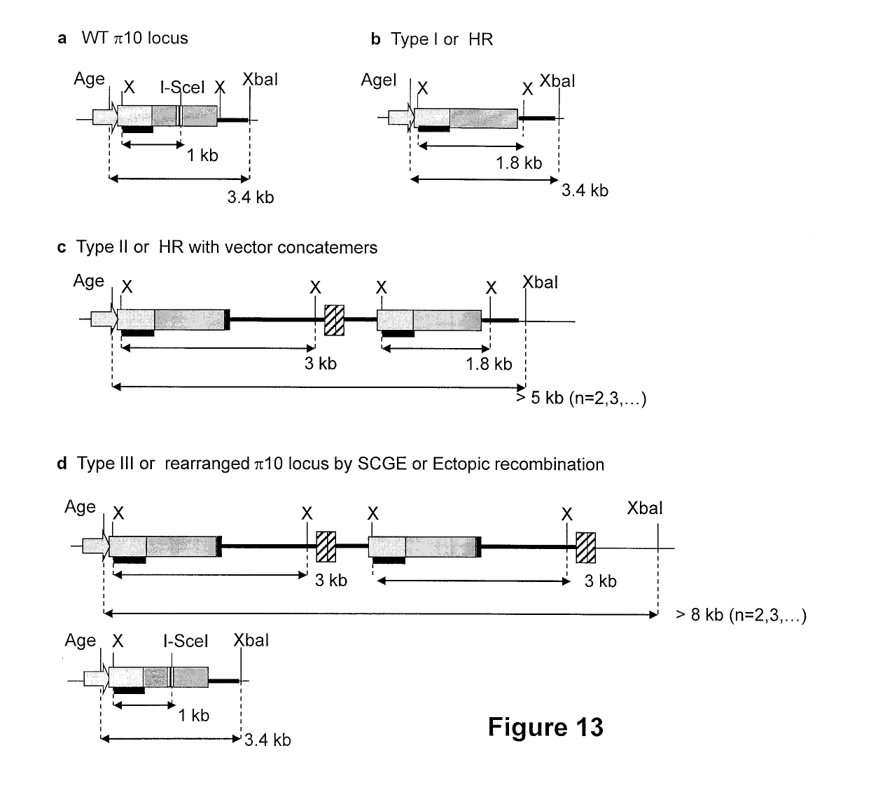

FIG. 13. Genetic structure of the π10 locus in Type I, II and III clones. Southern blot digestion profile for π10 locus. The position of restriction enzyme sites used for analysis is indicated.

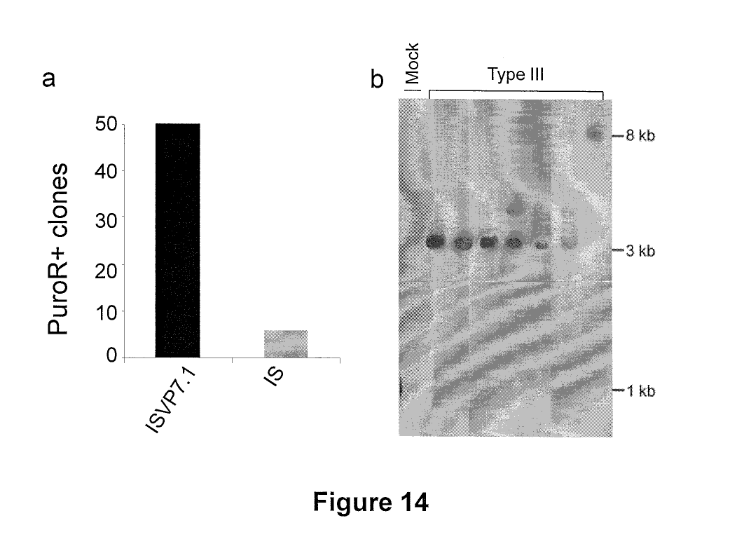

FIG. 14. Characterization of PuroR+ clones treated with IS containing particles. (a) 10e5 reporter cells were treated with IDLVs containing either ISVP7.1 or IS (RMA in cis). 72 hours after transduction, cells were treated with puromycin during 15 days and PuroR+ clones were counted. (b) Southern blot analysis of the π10 locus in Puro+ clones obtained with IS containing particles. Genomic DNA was digested with XmnI and I-SceI and hybridized with an internal EIE probe. Fragment sizes for: wt, 1 kb; Type III-1 kb+3 kb+8 kb.

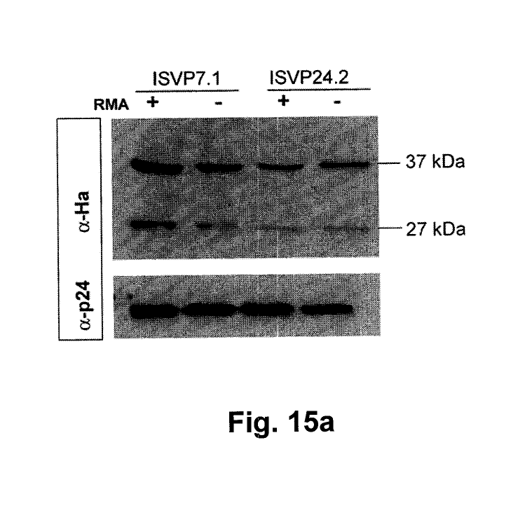

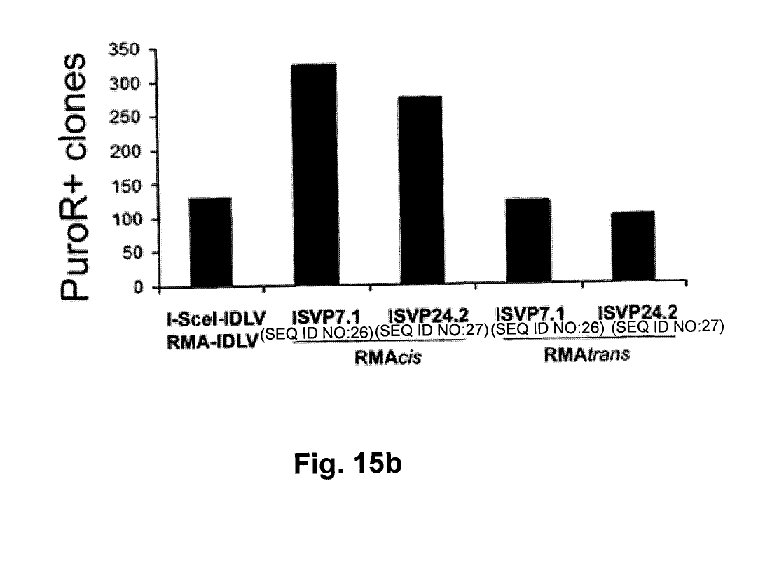

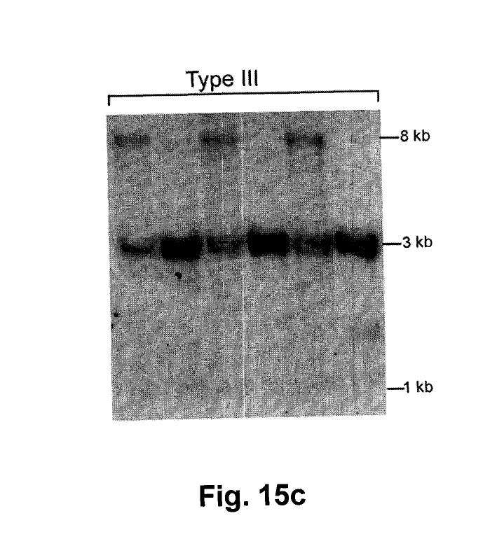

FIG. 15. I-SceI mediated π10 locus targeting using IDLVs defective for LEDGF/p75 interaction. (a) Western blot analysis of I-SceI containing NILV produced with a Q168C mutant Integrase. Concentrated particles (40-50 ng p24) were loaded on 10% SDS PAGE gels and probed with an antibody against the HA tag. Two bands corresponding to the cleaved (27 kDa) and non cleaved (37 kDa) proteins were obtained for both ISVP7.1 and ISVP24.2 containing particles. The same blot was probed with an antibody against HIV-1 Gag p24. (b) 10e5 reporter cells were treated with IDLVs prepared in the presence of ISVP7.1 or ISVP24.2, protein expression constructs. The RMA was brought either in cis or in trans. LV-I-SceI that encodes I-SceI (see FIG. 9) was used as a control. The data shown are representative of two independent experiments. The highest number of resistant clones was obtained when I-SceI was packaged as protein and RMA was brought in cis. (c) Genomic DNA from the PuroR+ clones was digested with XmnI and I-SceI and hybridized with the ³²P-labeled internal EIE probe.

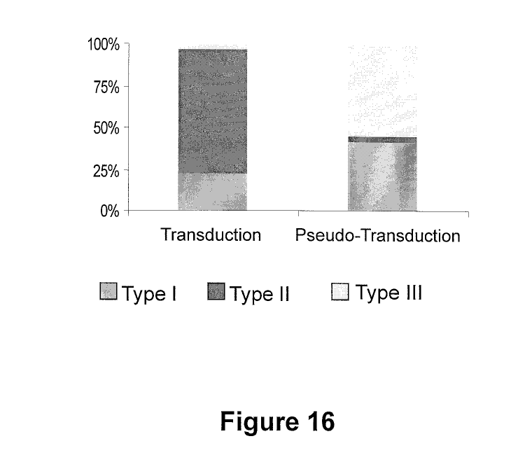

FIG. 16. Repartition of clone types upon recombination in PuroR+ clones.

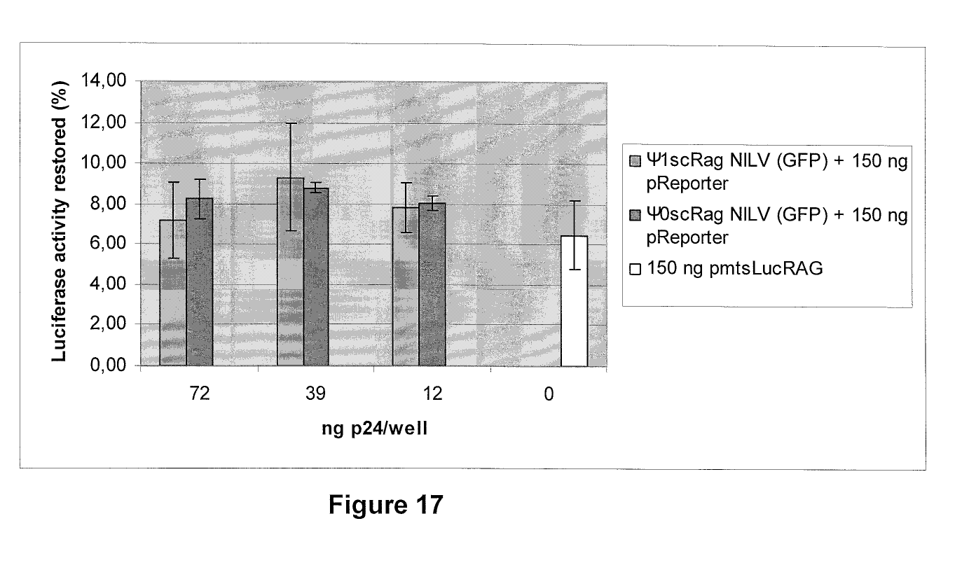

FIG. 17. Shows results from experiments in which a plasmid comprising an inactivated luciferase reporter gene that contains an internal sequence duplication interrupted by a Meganuclease recognition site, was transfected into human embryonic kidney cells (293 T) in a 96 well plate format (150 ng of expression plasmid and 2.10⁴ cells/well). Cells were then transduced with 12, 39 or 72 ng of p24 of NILV containing the meganuclease-Vpr fusion protein. Luciferase activity was measured after 72 hours. The Figure indicates that at all doses tested, the presence of the protein transducing NILVs were able to induce a luciferase signal over background, suggesting that functional MN have been introduced in the cells.

There will now be described by way of example a specific mode contemplated by the Inventors. In the following description numerous specific details are set forth in order to provide a thorough understanding. It will be apparent however, to one skilled in the art, that the present invention may be practiced without limitation to these specific details. In other instances, well known methods and structures have not been described so as not to unnecessarily obscure the description.

### EXAMPLE 1

### Materials and Methods

Non integrative lentiviral vectors (NILV) were produced as described by transient transfection of human embryonic kidney cells (293 T) with 4 packaging plasmids (HIV-1 gag-pol (FIG. 7C), HIV Tat (FIG. 7D), HIV Rev (FIG. 7A) and VSV-G (FIG. 7B), the sequence of HIV gag-pol (SEQ ID NO: 21), HIV Tat (SEQ ID NO: 22), HIV Rev (SEQ ID NO: 23) and VSV-G (SEQ ID No: 24) are also provided in the enclosed sequence listing) and a plasmid containing the recombinant lentiviral genome (transfer vector) which can be varied from one experiment to another, PHAGE-CMV-scRAG-HA and PHAGE-RMA_{—}0404 [09], see FIGS. 6, 7 and 8 for plasmid maps of constructs. 293 T cells were plated at 2.5×10e6 cells/plate on 15-cm Petri dishes in 24 ml medium (DMEM, 4.5 g/L glucose with Glutamax). 72 hours post-plating, the medium was changed and 2 hours later a transfection was done as follows: 12.5 μg pCLS-I-SceI-Vpr fusion, 10 μg PHAGE-RMA, 1 μg pHDMg-D64L, 1 μg p-Rev, 2.5 μl p-Tat, 7.9 μg pMDG, 0.1x TE 0.667 ml, 2.5M CaCL₂ 0.112 ml/H₂O 0.334 ml. Then, 1.140 ml of HBS2x buffer was added dropwise, and gently mixed. After 5 min at room temperature, the entire mixture added dropwise to the plate. 12 h later at 37° C. at 5% CO₂, the medium was changed, and the supernatants were collected three times each 12 hours and 13 ml of fresh medium was added to the plate. The collected supernatants were passed through 0.45 μm Millipore filter, supplemented with 20% sucrose and the ultracentrifugation was carried out in a Beckman centrifuge using a SW28 rotor at 4° C., 19500 rpm, 2 hours and re-suspended with 70 μl PBS-BSA 1% and stored at −80° C. until use.

The transfer vector contained either sequences homologous to the targeted locus (Repair MAtrix, RMA SEQ ID NO: 25, FIG. 8C) or a GFP expression cassette driven by the human PGK promoter. NILV physical particles (pp) were quantified using an HIV-1 Gag p24 ELISA, with 1 ng of p24 corresponding to 1.25×10⁷ pp. HIV p24 ELISA was done followed by manufacturer's protocol (Cell Biolabs, VP-108-HIV-p24). The concentrated lentivirus simple was diluted 10-1000 folds in culture medium, inactivated with TritonX-100 solution at 37° C. during 30 minutes and transferred into the micro-well strips coated with anti-p24 antibody and incubated ON at 4° C. After washing steps, the micro-well strips was incubated with diluted anti-FITC-conjugated anti-p24 monoclonal antibody during one hour, then washed and incubated with HRP-conjugated anti-FITC monoclonal antibody during one hour. After washing steps, the plate was incubated with substrate solution during 10 minutes. And finally the reaction was stopped with stop solution and the absorbance of each well was read on a spectrophotometer using 450 nm as the primary wave length.

Plasmids encoding Meganuclease::Vpr fusion proteins, expressed under the control of a CMV promoter were constructed. The fusion proteins include the following domains, from N- to C-terminal: a) a meganuclease amino acid sequence such as I-SceI or scRag1, b) a peptide from the influenza virus haemaglutinin used as a tag for protein immunodetection (HA tag) c) an HIV-1 protease cleavage site (see below) and d) the _{HIV-}_{LAI} Vpr amino acid sequence (position 1 to 96) (FIGS. 1 and 5). Two different protease cleavage sites from the HIV-1 Gag polyprotein are used [57]: 7/1, in the MN::Vpr1 fusion proteins and 24/2 in the MN::Vpr2 fusion proteins. The MN::Vpr0 fusion proteins contain no cleavage site. The following fusion proteins were used: fusion I-SceI-HA-p7/p1-vpr (SEQ ID NO: 26), fusion I-SceI-HA-p24/p2-vpr (SEQ ID NO: 27), Fusion I-SceI-HA-G-vpr (SEQ ID NO: 28) and Fusion I-SceI-HA-p7/p1-Δvpr 14-18 (SEQ ID NO: 29)

### EXAMPLE 2

### I-SceI::Vpr Fusion Proteins

NILV (non-integrating lentiviral vectors) were designed in order to simultaneously introduce a meganuclease (I-SceI) and a RMA into the CHO derived Pi-10.1 cell line, which contains an inactivated puromycine resistance gene (puro) [58]. The puro gene is interrupted by an I-SceI recognition sequence. When a promoter-less but functional puromycin resistance gene is introduced into Pi-10 cells following I-SceI mediated double strand break, homologous recombination occurs and results in gene repair. In this system, gene targeting is measured by the number of Puromycin resistant clones obtained after growing the cells in the presence of Puromycin. The recombination events in these resistant clones were then documented at the DNA level by PCR and Southern blot analysis.

Plasmids expressing I-SceI::Vpr fusions were transfected into human embryonic kidney cells (293 T), alone or along with all the lentiviral packaging functions and a transfer vector plasmid containing an RMA designed to repair the inactivated puro gene in Pi-10 cells.

Purified NILV and extracts from cells transfected with the fusion protein constructs alone were analysed by Western blot using an antibody against the HA tag epitope. NILV were prepared with I-SceI fusions including the 7/1 (Ψ1I-SceI(RMA)) (SEQ ID NO:29) or the 24/2 (Ψ2I-SceI(RMA)) (SEQ ID NO: 27) protease cleavage sites.

FIG. 1 shows that purified virions contain an immuno-reactive protein with the expected MW for the fusion protein (37 kDa) which is partially cleaved to liberate a 27.5 kDa species corresponding to the HA-tagged I-SceI. Both the 7/1 and 24/2 cleavage sites for the HIV protease are functional. The cleaved product appears only in the virions and is absent from the cell extract, indicating that, as expected, it is dependent on the presence of the viral protease and that the fusion proteins are indeed incorporated into the virions. The protease cleaved species is not detected in the control where a protein extract from 293 cells transfected with only I-SceI::Vpr2 is analysed (lane 3).

Next, the Ψ1I-SceI(RMA) and Ψ2I-SceI(RMA) NILV, which contained a packaged I-SceI::Vpr and a puro RMA (SEQ ID NO: 25) were used to transduce Pi-10 cells at doses of 100 to 500 pp per cell, under standard conditions for lentiviral vector mediated gene transfer. CHOpi10 cells were plated at 1.2×10e5 cells per well in 6-well plates in Keighn's medium (Invitrogen). The following day, 1 μl of 10 mg/ml polybrene (Chemicon) was added per well (1: 1000 final dilution) and virus-containing supernatant added as described above. After 72 h incubation, the cells were tripsinized, plated on 10 cm Petri dishes in a 7 ml medium. 12 h later, the medium was aspirated and replaced with the medium containing 10 mg/ml puromycin (Invitrogen). After 48 hours transduced cells were split into selective media containing 10 μg/ml puromycine. Resistant clones were scored after 2 weeks and isolated for further analysis. FIG. 2 shows the number of resistant clones obtained, demonstrating that the NILV are able to deliver functional I-SceI to the target cells.

In a similar experiment, the amount of HR obtained with NILVs containing both I-SceI and the RMA was compared to that obtained with two separate vectors, one with I-SceI and a GFP containing genome (ΨI-SceI(GFP) or Ψ2I-SceI(GFP)) and one bringing the RMA alone in trans. FIG. 3 indicates that although recombination can be detected in the trans situation, it is an order of magnitude higher when all components are brought in the same viral particle.

In a control experiment, HR was obtained using two different NILV carrying the RMA and the I-SceI cDNA respectively at 1000 to 5000 pp/cell. The frequency of HR was in the same range as when the I-SceI::Vpr containing NIVL were used.

Puromycin resistant cells were analysed at the recombination locus using a PCR assay, providing further evidence of bona fide gene targeting using the meganucleases delivered in this new manner to the target cells (FIG. 4).

The following primers were used for the PCR:

Puro Fw
(SEQ ID NO: 30)
5′CCGCCACCATGACCGAGTACAA3′,
Puro-Isc Fw
(SEQ ID NO: 31)
5′ACGAAGTTATGGTCACCGAG3′
and
Puro Rev
(SEQ ID NO: 32)
5′CTCGTAGAAGGGGAGGTTGCG3′.

### EXAMPLE 3

### scRag::Vpr Fusion Proteins

NILV were designed in order to simultaneously introduce an engineered single chain meganuclease derived from I-CreI (scRag-1) (SEQ ID NO: 33) and an RMA see FIG. 8C, into the CHO derived Pi-10.1.10.Rag cell line which contains an inactivated β-galactosidase gene (lacZ) [58]. The lac Z gene is interrupted by a scRag1 recognition sequence. When a promoter-less repair matrix containing uninterrupted lacZ sequences is introduced into the pi-10.1.10.Rag cells, following scRag1 mediated double strand break, homologous recombination occurs and results in gene repair. In this system, gene targeting is measured by the number of β-galactosidase positive cells obtained 72 hours after introduction of the meganuclease and RMA.

Plasmids expressing scRag1:: Vpr fusions with or without the 7/1 protease cleavage site were transfected into human embryonic kidney cells (293 T), alone or along with all the lentiviral packaging functions and a transfer vector plasmid containing an RMA designed to repair the inactivated lacZ gene in pi-10.1.10.Rag cells.

Purified NILV and extracts from cells transfected with the fusion proteins alone were analysed by Western blot using an antibody against the HA tag epitope. FIG. 5 shows that purified virions contain an immuno-reactive protein with the expected MW for the fusion protein (53 kDa) which is partially cleaved to liberate a 41 kDa species corresponding to the HA-tagged scRag1.

In the presence of the 7.1 cleavage site, the cleaved product appears only in the virions and is absent from the cell extract, indicating that as expected it is dependent on the presence of the viral protease and that the fusion proteins are indeed incorporated into the virions.

NILV containing the meganuclease and an RMA (lacZ) genome were used to transduce pi-10.1.10.Rag cells at doses of 100 or 500 pp per cell, using standard conditions for lentiviral vector mediated gene transfer. After 72 hours cells were fixed and histochemically stained for lacZ activity. Table I shows the number of lacZ positive cells scored. It indicates that the Y1scRag::Vpr (RMA.lacZ) vector is able to co-deliver a repair matrix and a functional site specific MN to target cells. It also shows that the protease cleavage site is dispensable in this particular case, although HR efficiency tends to be lower.

Table I also shows the results of a control experiment in which HR was obtained using two different NILV carrying the RMA and the scRag1 cDNA (NILV (RMA.lacZ) and NILV (scRag), respectively). Higher levels of recombination are found under these conditions.

TABLE I

| Vector 1 | Particles/Cell | Vector2 | Particles/cell | lacZ+ve cells |
|---|---|---|---|---|
| NILV (scag) | 100 pp | NILV | 100 pp | 1216 + 956 |
| | | (RMA.lacZ) | | |
| NILV (scag) | 500 pp | NILV | 100 pp | 1608 + 1640 |
| | | (RMA.lacZ) | | |
| NILV (scag) | 500 pp | | | 0 + 0 |
| NILV (scag) | 100 pp | | | 1 + 0 |
| Ψ1scRag | 100 pp | | | 9 + 6 |
| (RMA.lacZ) | | | | |
| Ψ1scRag | 500 pp | | | 20 + 25 |
| (RMA.lacZ) | | | | |
| Ψ0scRag | 500 pp | | | 3 + 3 |
| (RMA.lacZ) | | | | |
| Ψ0scRag | 100 pp | | | 11 + 11 |
| (RMA.lacZ) | | | | |

### EXAMPLE 4

### I-SceI Mediated CHOπ10 Locus Targeting by Integration Deficient Lentiviral Vectors IDLV Delivery

The efficiency of I-SceI mediated homologous recombination by lentiviral delivery was evaluated in CHOπ10 cells using Integration Deficient

Lentiviral Vectors (IDLV) encoding I-SceI and a Recombination Matrix (RMA) with a functional puromycin resistance gene (FIG. 9a).

IDLV were obtained by the introduction of a mutation of residue D64 in the HIV-1 Integrase gene, this mutation changes the DDE catalytic triad of the integrase.

When the reporter cells were transduced with increasing doses of IDLVs, the number of puromycin resistant clones increased in a dose dependent manner. The gene targeting efficiency was found to be around 1%, as estimated by counting the number of puromycin resistant clones. When the dose of IDLV-I-SceI was kept constant, while the dose of RMA encoding lentivirus was increased, a lower recombination rate was obtained for all experimental points, indicating that the amount of I-SceI was limiting (FIG. 9b).

I-SceI mediated CHOπ10 locus targeting by IDLVs was confirmed by PCR and Southern blot analysis (FIG. 9c). In randomly isolated and amplified clones the Puromycin cassette was present at the targeted locus either as a single copy (type I clones) or in the form of head-to-tail or head-to-head repeats, likely to occur through intermolecular recombination or ligation of the lentiviral LTR sequences (type II clones).

### EXAMPLE 5

### I-SceI Protein Incorporation into Viral Particles

The system described above requires the use of multiple vectors, expressed for several days, which may cause toxicity due to possible non-specific meganuclease cleavage activity. For this reason the inventors have combined lentivirus based nucleic acid delivery with protein transducing technology to avoid prolonged chromosome exposure to DSB. In our system a single IDLV is used to introduce the recombination matrix and the I-SceI as protein associated with viral particles.

To achieve the incorporation of the I-SceI into lentiviral particles, different expression constructs with the I-SceI meganuclease fused to the lentivirus accessory protein Vpr were generated.

In all constructs, I-SceI carried a C-terminal HA tag and was fused in-frame to the N-terminus of the viral accessory protein Vpr or ΔVpr (a Vpr fragment comprising just amino acids 14 to 88 of the full length Vpr protein). Two different cleavage sites for the HIV protease (p7/1 or p24/2) were introduced upstream of Vpr, in order to generate a Vpr-free meganuclease after processing inside the virion (FIG. 10a). To characterize these fusion constructs named ISVP7.1 (I-SceI-Ha-p7/1-Vpr), ISVP24.2 (I-SceI-Ha-p24/2-Vpr) and ISΔVP7.1 (I-SceI-Ha-p7/1-ΔVpr), the corresponding plasmids were transfected in HEK293-T cells. Cell lysates were prepared and analysed by Western blot with an antibody against HA after 32 hours.

The expected bands of 37 kDa or 33 kDa were obtained corresponding to ISVP7.1, ISVP24.2 or ISΔVP7.1 respectively (FIG. 11a). I-SceI containing particles were generated in the presence or the absence of a transfer vector containing the RMA, purified and analysed by Western blot. Each I-SceI fusion protein was correctly packaged into lentiviral virions, processed by the viral protease and encapsidated into VSV-G pseudotyped virions. High concentrations of p24 were obtained with each construct indicating that Vpr based incorporation of foreign proteins does not affect the production and release of Gag proteins. The Western blot analysis of purified virions revealed two bands of comparable intensity corresponding to the non-cleaved and cleaved fusion proteins. A lower level of cleavage was obtained with ΔVpr.

To ensure that the detected protein is localised in the particle and not trapped on the viral membrane, ISVP24.2 containing particles were treated with increasing doses of Proteinase K (0.02 to 0.1 mg/ml) and analysed by Western Blot using antibodies against HA or VSV_G. The VSV-G protein present on the virion surface was readily digested whether most of the I-SceI containing fusions were resistant to digestion (FIG. 11b).

### EXAMPLE 6

### Targeting of CHOπ10 Locus by I-SceI Containing Particles

To evaluate the activity of the packaged meganuclease CHOπ10 reporter cells were treated with I-SceI containing viral particles carrying a puromycin repair matrix (IDLV-RMA cis). Alternatively, the RMA was brought by a separate lentivirus (IDLV-RMA trans) (FIG. 12a). The results show that the virion incorporated I-SceI protein can drive HR three times more efficiently than when conventional lentiviral delivery is used. The efficiency was significantly decreased when the recombination matrix was brought in trans, indicating that the presence of both the

RMA and the meganuclease in the same particle was facilitating recombination.

A lower HR efficiency was obtained with ISΔVP7.1 containing particles, possibly because of the less efficient processing of this fusion by the viral protease. To determine whether Vpr alone could have an HR enhancing effect, the cells were transduced by Vpr containing particles (VP), with the RMA being either brought in cis or in trans. Puromycin resistant clones were indeed obtained, when the recombination matrix was brought in cis.

In a second control experiment the I-SceI protein, without Vpr was expressed during vector production and viral particles were analysed by Western blot. A low level of I-SceI was found to be associated to the particles in the absence of Vpr. These low amounts of virion-associated meganuclease resulted in targeted Puromycin resistant clones in the CHOπ10 assay (FIG. 14).

Targeting of the π10 locus was confirmed on randomly isolated and amplified Puromycin resistant clones by PCR (data not shown) and Southern blot analysis (FIG. 12 b,c).

Three different digestion profiles were obtained including 41% type I and 3% type II as described above (FIG. 13). A third type of pattern was observed in about half of the clones analysed (FIG. 16). These type III clones contained the recombined structure, together with a retained copy of the original, I-SceI containing locus. These clones could have been generated by mechanisms such as sister chromatid exchange or ectopic recombination (Johnson and Jasin, 2000, EMBO J, 19 (13) 3398-3407; Mangerich et al., 2009, Transgenic Res, 18 (2), 261-279). Type III clones were only seen when the RMA was brought in cis in an I-SceI containing viral particle. It was also observed in the few clones obtained in the absence of fusion to Vpr, suggesting that Vpr is not a major determinant of these unconventional recombination events.

Type II or III targeting events are associated with the formation of vector concatemers, presumably through LTR ligation. The HIV integrase can act as an endonuclease that cleaves LTR junctions and therefore it was decided to investigate whether using a mutant integrase with an intact catalytic site could help avoid these structures. NILV were produced with a Q168C integrase mutant which is has lost its ability to bind LEDGF/p75 and is therefore inactive. No difference in the structure of the targeted π10 locus was documented when using these particles (FIG. 15).

### EXAMPLE 7

### Activity of scRag::Vpr Fusion Proteins

NILV were designed in order to introduce proteins form of single chain meganuclease derived from I-CreI (scRag-1) into 293 T cells. The induction of recombination by these proteins was measured using a luciferase reporter assay. This assay uses an inactivated luciferase reporter gene that contains an internal sequence duplication interrupted by a MN recognition site. Cleavage of the target site induces tandem-repeat recombination, thereby restoring a functional luciferase gene. A plasmid expressing this reporter was transfected into human embryonic kidney cells (293 T) in a 96 well plate format (150 ng of expression plasmid and 2.10⁴ cells/well). Cells were then transduced with 12, 39 or 72 ng of p24 of NILV containing the meganuclease-Vpr fusion protein. Luciferase activity was measured after 72 hours.

FIG. 17 shows that at all doses tested, the presence of the protein transducing NILVs were able to induce a luciferase signal over background, suggesting that functional MN have been introduced in the cells.

### EXAMPLE 8

### Conclusions

The inventors have produced replication deficient lentiviral particles based on HIV-1, which are equipped with chimeric proteins composed of a meganuclease fused to the Vpr viral protein. The meganuclease can be liberated if a cleavage site for the lentiviral protease is present between the two domains of the fusion protein. The same lentiviral particles may also package a recombinant genome containing a repair matrix for homologous recombination.

Based on the same principle, a variety of vectors could be assembled to introduce active endonuclease into cells in the absence of nucleic acids encoding them. Cells can be of any type, established as lines or in primary culture, actively dividing or not. In principle, tissues such as the skin, the liver, the retina or the brain could be directly targeted by in vivo injection of the modified lentiviral vectors.

### REFERENCES

1. Paques, F. and P. Duchateau, Meganucleases and DNA double-strand break-induced recombination: perspectives for gene therapy. Curr Gene Ther, 2007. 7(1): p. 49-66.

2. Chevalier, B.S. and B.L. Stoddard, Nucleic Acids Res., 2001, 29, 3757-3774.

3. Chevalier, et al, Nat. Struct. Biol., 2001, 8, 312-316

4. Chevalier et al, J. Mol. Biol., 2003, 329, 253-269

5. Spiegel et al, Structure, 2006, 14, 869-880

6. Moure et al, J. Mol. Biol., 2003, 334, 685-69

7. Silva et al., J. Mol. Biol., 1999, 286, 1123-1136

8. Bolduc et al., Genes Dev., 2003, 17, 2875-2888

9. Ichiyanagi et al., J. Mol. Biol., 2000, 300, 889-901

10. Moure et al., Nat. Struct. Biol., 2002, 9, 764-770

11. Chevalier et al., Mol. Cell., 2002, 10, 895-905

12. Epinat et al., Nucleic Acids Res, 2003, 31, 2952-6

13. WO 2003/078619

14. WO 2004/031346

15. Seligman et al., Genetics, 1997, 147, 1653-1664

16. Sussman et al., J. Mol. Biol., 2004, 342, 31-41

17. WO 2006/097784

18. WO 2006/097853

19. WO 2007/060495

20. WO 2007/049156

21. Arnould et al., J. Mol. Biol., 2006, 355, 443-458

22. Rosen et al., Nucleic Acids Res., 2006, 34, 4791-4800

23. Smith et al., Nucleic Acids Res., 2006, 34, e149

24. Doyon et al., J. Am. Chem. Soc., 2006, 128, 2477-2484

25. Gimble et al., J. Mol. Biol., 2003, 334, 993-1008

26. Ashworth et al., Nature, 2006, 441, 656-659

27. WO 2006/097854

28. WO 2007/034262

29. WO 2007/049095

30. WO 2007/057781

31. WO 2004/067736

32. Chames et al., Nucleic Acids Res., 2005, 33, e178

33. WO2007/093918

34. WO2008/010093

35. WO2008/059382

36. WO2008/102274

37. WO2008/152523

38. WO2009/13622

39. WO200901961440. Cornu, T. I. and T. Cathomen, Targeted genome modifications using integrase-deficient lentiviral vectors. Mol Ther, 2007. 15(12): p. 2107-13.

41. Lombardo, A., P. Genovese, C. M. Beausejour, S. Colleoni, Y. L. Lee, K. A. Kim, D. Ando, F. D. Urnov, C. Galli, P. D. Gregory, M. C. Holmes, and L. Naldini, Gene editing in human stem cells using zinc finger nucleases and integrase-defective lentiviral vector delivery. Nat Biotechnol, 2007. 25(11): p. 1298-306.

42. Miller, D. G., L. M. Petek, and D. W. Russell, Human gene targeting by adeno-associated virus vectors is enhanced by DNA double-strand breaks. Mol Cell Biol, 2003. 23(10): p. 3550-7.

43. Porteus, M. H., T. Cathomen, M. D. Weitzman, and D. Baltimore, Efficient gene targeting mediated by adeno-associated virus and DNA double-strand breaks. Mol Cell Biol, 2003. 23(10): p. 3558-65.

44. Asokan, A., J. S. Johnson, C. Li, R. J. Samulski, Bioluminescent virion shells: new tools for quantitation of AAV vector dynamics in cells and live animals. Gene Ther, 15:1618 (2008).

45. Vellinga, J., M. J. W. E. Rabelink, S. J. Cramer, D. J. M. van den Wollenberg, H. Van der Meulen, K. N. Leppard, F. J. Fallaux, R. C. Hoeben, Spacers Increase the Accessibility of Peptide Ligands Linked to the Carboxyl Terminus of Adenovirus Minor Capsid Protein IX. J. Virol., 78:3470-3479 (2004).

46. Muratori, C., P. D'Aloja, F. Superti, A. Tinari, N. Sol-Foulon, S. Sparacio, V. Bosch, O. Schwartz, M. Federico, Generation and characterization of a stable cell population releasing fluorescent HIV-1-based Virus Like Particles in an inducible way. BMC Biotechnol, 6:52 (2006).

47. Yamauchi, Y., K. Kiriyama, N. Kubota, H. Kimura, J. Usukura, Y. Nishiyama, The UL14 Tegument Protein of Herpes Simplex Virus Type 1 Is Required for Efficient Nuclear Transport of the Alpha Transinducing Factor VP16 and Viral Capsids. J. Virol., 82:1094-1106 (2008).

48. Zhang, C., A. Qin, H. Chen, X. Deng, Y. Su, K. Qian, MDV-1 VP22: a transporter that can selectively deliver proteins into cells. Archives of

Virology, 154:1027 (2009).

49. Qi, M., C. Aiken, Nef enhances HIV-1 infectivity via association with the virus assembly complex. Virology, 373:287 (2008).

50. Malim, M. H., M. Emerman, HIV-1 Accessory Proteins—Ensuring Viral Survival in a Hostile Environment. Cell Host & Microbe, 3:388-398 (2008).

51. Zufferey, R., D. Nagy, R.J. Mandel, L. Naldini, D. Trono, Multiply attenuated lentiviral vector achieves efficient gene delivery in vivo. Nat Biotech, 15:871-875 (1997).

52. Jenkins, Y., O. Pornillos, R. L. Rich, D. G. Myszka, W. I. Sundquist, M. H. Malim, Biochemical Analyses of the Interactions between Human Immunodeficiency Virus Type 1 Vpr and p6Gag. J. Virol., 75:10537-10542 (2001).

53. Dispensable role of the Human-Immunodeficiency-Virus Type-2 Vpx protein in viral replication, Marcon L, Michaels F, Hattori N, Fargnoli K, Gallo R C, Franchini G. Journal of Virology 65 (7): 3938-3942 JUL 1991

54. Vpx and Vpr proteins of HIV-2 up-regulate the viral infectivity by a distinct mechanism in lymphocytic cells, Ueno F, Shiota H, Miyaura M, Yoshida A, Sakurai A, Tatsuki J, Koyama A H, Akari H, Adachi A, Fujita M. Microbes and Infection 5 (5): 387-395 APR 2003.

55. Vink, C. A., H. B. Gaspar, R. Gabriel, M. Schmidt, R. S. McIvor, A. J. Thrasher, and W. Qasim, Sleeping Beauty Transposition From Nonintegrating Lentivirus. Mol Ther, 2009.

56. Keravala, A., S. Lee, B. Thyagarajan, E. C. Olivares, V. E. Gabrovsky, L. E. Woodard, and M. P. Calos, Mutational Derivatives of PhiC31 Integrase With Increased Efficiency and Specificity. Mol Ther, 2008. 17(1): p. 112-120.

57. Serio, D., T. A. Rizvi, M. Cartas, V. S. Kalyanaraman, I. T. Weber, H. Koprowski, A. Srinivasan, Development of a novel anti-HIV-1 agent from within: effect of chimeric Vpr-containing protease cleavage site residues on virus replication. Proc Natl Acad Sci USA, 94:3346-51 (1997).

58. Arnould, S., C. Perez, J.P. Cabaniols, J. Smith, A. Gouble, S. Grizot, J. C. Epinat, A. Duclert, P. Duchateau, and F. Paques, Engineered I-CreI derivatives cleaving sequences from the human XPC gene can induce highly efficient gene correction in mammalian cells. J Mol Biol, 2007. 371(1): p. 49-65.

59. U.S. Pat. No. 7,402,436.

60. U.S. 2005/0266565

61. Johnson and Jasin, 2000, EMBO J, 19 (13) 3398-3407.

62. Mangerich et al., 2009, Transgenic Res, 18 (2), 261-279).

1. A fusion protein comprising:
a meganuclease, which recognizes and cleaves a specific DNA target sequence, and
at least one viral peptide selected from the group consisting of Vpr, Vpx, a fragment of Vpr, a fragment of Vpx, a derivative of Vpr, and a derivative of Vpx;
wherein the fusion protein associates with a Lentivirus vector particle, and
the fusion protein further recognizes and cleaves the specific DNA target sequence in vivo, following transduction into a host cell.

2. The fusion protein of claim 1, further comprising a Lentivirus protease cleavage site, positioned between the meganuclease and the vital peptide.

3. The fusion protein of claim 1, further comprising a detectable tag.

4. The fusion protein of claim 1, wherein the meganuclease is at least one meganuclease selected from the group consisting of I-SceI, I-ChuI, I-Cre I, I-DmoI, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, I-MsoI, PI-Tsp I, and variants or derivatives thereof.

5. The fusion protein of claim 1, wherein the viral peptide comprises SEQ ID NO: 15.

6. A polynucleotide, which encodes the fusion protein of claim 1.

7. A lentiviral vector comprising the fusion protein of claim 1.

8. The lentiviral vector of claim 7, further comprising a DNA molecule encoding a repair matrix (RMA).

9. The lentiviral vector of claim 7, further comprising a non-functional lentiviral integrase.

10. The lentiviral vector of claim 7, further comprising an exogenous surface antigen.

11. A method of altering a genomic DNA sequence of a target cell in vitro, comprising providing the polynucleotide of claim 6 to the target cell.

12. A medicament comprising the fusion protein of claim 1.

13. A host cell, obtained by a process comprising modifying the host cell with the fusion protein of claim 1.

14. A non-human transgenic animal, comprising the host cell of claim 13.

15. The fusion protein of claim 2, wherein the Lentivirus protease cleavage site is an HIV protease cleavage site.

16. The fusion protein of claim 1, further comprising a Nuclear Localization Signal (NLS).

17. The fusion protein of claim 16, wherein the NLS is at an NH₂ terminus, a COOH terminus, or both of the meganuclease.

18. The fusion protein of claim 16, wherein the NLS comprises a short sequence of positively charged amino acids.

19. The lentiviral vector of claim 8, wherein the RMA is integrated into a viral genome.

20. The lentiviral vector of claim 8, wherein the RMA is an episomal DNA molecule.
